SEQUENCE LISTING (4 sequences)

SEQ ID NO: 1 - DNA, 1446 bp, Homo sapiens
atgatgctcaagtctatcacagaaagctttgccacagcaatccatggcttgaaagtgggacacctgacagatcgtgttattcagaggagcaagaggatgattctagacactctgggtgctgggttcctgggaaccactacggaagtgtttcacatagccagccaatatagcaagatctacagttccaacatatccagcactgtttggggtcagccagacatcaggctcccgcccacatatgctgcttttgtgaacggtgtggctattcactccatggattttgatgacacgtggcaccctgccacccacccttctggggctgtccttcctgtcctcacagctttagcagaagccctgccaaggagtccaaagttttctggccttgacctgctgctggctttcaatgttggtattgaagtgcaaggccgattactgcatttcgccaaggaggccaatgacatgccaaagagattccatcccccttccgtggtaggaacgttgggtagtgctgctgctgcatccaagtttttaggacttagctcgacaaagtgccgagaagctctggccattgctgtttcccatgctggggcacccatggccaatgctgccacccagaccaagcccctccacattggcaatgctgccaagcatgggatagaagctgcatttttggcaatgttgggtctccaaggaaacaagcaggtcttggacttggaggcaggatttggggccttttatgccaactattccccaaaagtccttccaagcatagcttcctacagttggctgctggaccagcaggacgtggcctttaagcgttttcctgcacatttatctacccactgggtggcagacgcagctgcatctgtgagaaagcaccttgtagcagagagagccctgcttccaactgactacattaagagaattgtgctcaggataccaaatgtccagtatgtaaacaggccctttccagtttcggagcatgaagcccgtcattcattccagtatgtggcctgtgccatgctgcttgatggtggcatcactgtcccctcattccatgaatgccagatcaacaggccacaggtgagagagctgctcagtaaggtggagctggagtaccctccggacaacttgccaagcttcaacatactgtactgtgaaataagtgtcaccctcaaggatggagccaccttcacagatcgctctgataccttctatgggcactggagaaaaccactgagccaggaggacctagaggaaaagttcagagccaatgcctccaagatgctgtcctgggacacagtggaaagccttataaagatagtcaaaaatctagaagacctagaagactgttctgtgttaactacacttctcaaaggaccctctccaccagaggtagcttcaaactctccagcatgtaataattctatcacaaatctctcctga

SEQ ID NO: 2 - protein, 481 aa, Homo sapiens
MMLKSITESFATAIHGLKVGHLTDRVIQRSKRMILDTLGAGFLGTTTEVFHIASQYSKIYSSNISSTVWGQPDIRLPPTYAAFVNGVAIHSMDFDDTWHPATHPSGAVLPVLTALAEALPRSPKFSGLDLLLAFNVGIEVQGRLLHFAKEANDMPKRFHPPSVVGTLGSAAAASKFLGLSSTKCREALAIAVSHAGAPMANAATQTKPLHIGNAAKHGIEAAFLAMLGLQGNKQVLDLEAGFGAFYANYSPKVLPSIASYSWLLDQQDVAFKRFPAHLSTHWVADAAASVRKHLVAERALLPTDYIKRIVLRIPNVQYVNRPFPVSEHEARHSFQYVACAMLLDGGITVPSFHECQINRPQVRELLSKVELEYPPDNLPSFNILYCEISVTLKDGATFTDRSDTFYGHWRKPLSQEDLEEKFRANASKMLSWDTVESLIKIVKNLEDLEDCSVLTTLLKGPSPPEVASNSPACNNSITNLS

SEQ ID NO: 3 - DNA, 1467 bp, Mus musculus
atgatgctcaagtctgtcacagagagctttgctggtatgattcacggcttgaaagtgaaccacctgacagatggtatcattcggaggagcaagaggatgatcctggattctctgggcgttggcttcctggggacaggcacagaagtgttccataaagtcacccaatatagtaaaatctacagttccaacacctccagcactgtttggggtcgaccagacttcaggctcccaccgacatatgctgcttttgttaatggtgttgctgttcactccatggattttgatgacacatggcaccctgccacccacccttctggggctgtcctacctgtcctcacagctctatcggaagccctgcctcagactcccaagttttctggcctcgacctgctgctggcgttcaacgttggtattgaagtacagggacgattaatgcacttctccaaggaagccaaagacataccaaagagattccaccctccctctgtggtggggactctgggaagtgctgctgctgcgtccaagtttctggggctcagcttgacaaagtgccgcgaggcattggctattgctgtttcccacgcaggggcacccatagcgaacgctgccactcagactaagccccttcatattggcaatgcagccaagcatgggatggaagccacgtttctggcaatgctgggcctccaaggaaacaaacagatcttggacctggggtcagggttcggtgccttctatgccaactactcccccgaagaccttccaagcctggattctcacatctggctgttggaccagcaggatgtggcctttaagagcttcccggcacatctggctacccactgggtggcagatgcagctgcagccgtgagaaagcaccttgtgacaccagaaagagccctgttccctgctgaccacatcgagagaatcgtgctcaggatccctgacgtccagtacgtaaacaggcccttcccggactcagagcatgaagcccgtcattctttccagtatgtggcctgtgcctcgctgctcgacggtagcatcactgtcccatccttccacagccagcaggtcaataggcctcaggtgagagagttgctcaagaaggtgaagctggagcatcctcctgacaacccgccaagcttcgacacgctatactgtgaaataagcatcactctaaaggacgggaccactttcaccgagcgctctgacaccttctatggtcactggaggaaaccactgagccaggaagatctgcgcaacaagttccgagccaatgcctcaaagatgctatgcagggacacggtggaaagccttataacggtagtagaaaagctagaagacctagaagactgctctgtgctaaccagacttctgaaaggaccctctgtccaagatgaagcttcaaaactatccagcatgtcctcattcgatcacacaacgttgcccaggtttaccaatatctaa

SEQ ID NO: 4 - protein, 488 aa, Mus musculus
MMLKSVTESFAGMIHGLKVNHLTDGIIRRSKRMILDSLGVGFLGTGTEVFHKVTQYSKIYSSNTSSTVWGRPDFRLPPTYAAFVNGVAVHSMDFDDTWHPATHPSGAVLPVLTALSEALPQIPKFSGLDLLLAFNVGIEVQGRLMHFSKEAKDIPKRFHPPSVVGTLGSAAAASKFLGLSLTKCREALAIAVSHAGAPIANAATQTKPLHIGNAAKHGMEATFLAMLGLQGNKQILDLGSGFGAFYANYSPEDLPSLDSHIWLLDQQDVAFKSFPAHLATHWVADAAAAVRKHLVTPERALFPADHIERIVLRIPDVQYVNRPFPDSEHEARHSFQYVACASLLDGSITVPSFHSQQVNRPQVRELLKKVKLEHPPDNPPSFDTLYCEISITLKDGTTFTERSDTFYGHWRKPLSQEDLRNKFRANASKMLCRDTVESLITVVEKLEDLEDCSVLTRLLKGPSVQDEASKLSSMSSFDHTTLPRFTNI

(19) United States
(12) Patent Application Publication
(10) Pub. No.: US 2016/0060660 A1
(43) Pub. Date: Mar. 3, 2016

(54) BIOTECHNOLOGICAL PRODUCTION OF ITACONIC ACID

(71) Applicant: UNIVERSITÉ DU LUXEMBOURG, Luxembourg (LU)

(72) Inventors: Karsten Hiller, Esch-sur-Alzette (LU); Thekla Cordes, Esch-sur-Alzette (LU); Alessandro Michelucci, Esch-sur-Alzette (LU)

(21) Appl. No.: 14/782,533

(22) PCT Filed: Apr. 4, 2014
(86) PCT No.: PCT/EP2014/056840

§ 371 Date: Oct. 5, 2015

(30) Foreign Application Priority Data
Apr. 5, 2013 (EP) 13001749.4
Apr. 22, 2013 (LU) LU92184

(51) Int. Cl. C12P 7/44; C12N 9/88

(52) U.S. Cl. CPC C12P 7/44; C12N 9/88; C12Y 401/01006

(57) ABSTRACT

The present invention provides a novel method and means of producing itaconic acid based on the use of the mammalian Immune response gene 1 (Irg1) and variants to express an enzyme which converts cis-aconitic acid to itaconic acid. The method includes cultivation a host cell comprising the Igr1 gene or variants thereof and obtaining itaconic acid.

### FIELD OF THE INVENTION

The present invention relates to the production of itaconic acid, and more specifically, a bio-based production of itaconic acid.

### BACKGROUND OF THE INVENTION

Itaconic acid is an organic acid, also known as methylene succinic acid. It is an unsaturated dicarbonic acid in which one carboxyl group is conjugated to a methylene group. Itaconic acid is used in the manufacture of complex organic compounds. It is used in various reactions, such as salt formation with metals, esterification with alcohols, production of anhydride, addition reactions, and polymerization. The industrial versatility of itaconic acid and its reaction compounds is reflected in a wide range of applications. Itaconic acid is used in the industrial synthesis of resins such as polyesters, plastics, and artificial glass, and in the preparation of bioactive compounds in the agriculture, pharmacy, and medicine sectors (55, 21).

Itaconic acid was originally discovered as a product of pyrolytic distillation of citric acid (21). Later it was observed that some microorganisms like Aspergillus sp., Ustilago zeae, Ustilago maydis, Candida sp. or Rhodotorula sp. are able to synthesize this organic acid (53).

Itaconic acid can be produced both chemically and biotechnologically. However, no chemical process has been able to compete with the biological route (55, 56). Since the 1940s various Aspergillus species, like Aspergillus itaconicus and Aspergillus terreus, have been known as producers for the bio-based production of itaconic acid. In Aspergillus terreus, itaconic acid is formed by an allylic rearrangement and decarboxylation of cis-aconitic acid, an intermediate of the tricarboxylic acid (TCA) cycle (53). The reaction is catalyzed by the fungal enzyme cis-aconitic acid decarboxylase (CAD). Today itaconic acid is mainly achieved by the fermentation of sugars using Aspergillus terreus, which is the most frequently used production host of itaconic acid, because it can secrete high amounts (up to 80-86 g/L) of itaconic acid to the media (53).

Currently, there are still many problems associated with the production of itaconic acid, including low production rate and high cost (53, 54). Considering the broad industrial demand for itaconic acid, there is a continuing need for improved methods of producing itaconic acid.

### SUMMARY OF THE INVENTION

The present invention is based on surprising finding that mammalian immune response gene 1 (Irg1) gene, also called “immunoresponsive gene 1” herein (both terms can be used interchangeably) can be exploited in the process of itaconic acid production.

Although itaconic acid has been detected in mammalian cells, where it was found in macrophage-derived cells, the specific gene encoding this enzymatic activity was not known (19). The inventors have successfully identified Irg1 as the gene which encodes an enzyme that catalyzes the production of itaconic acid in mammalian cells. More precisely, Irg1 has been found to encode the enzyme that catalyzes the decarboxylation of the TCA cycle intermediate cis-aconitate to itaconic acid. The comparable function of Irg1 and CAD suggests that the biosynthesis of itaconic acid is evolutionary conserved. Hence the findings of the inventors disclose the unexpected possibility to produce itaconic acid by expressing the mammalian Irg1 gene or variants thereof in a heterologous host cell.

In a first aspect, the present invention provides a method of producing itaconic acid, comprising expressing a nucleic acid molecule encoding a Irg1 gene or a variant thereof in a host cell, more preferably, in a non-human host cell such as non-mammalian host cell.

In a preferred embodiment, present invention provides a method for the production of itaconic acid, comprising
(i) expressing in a non-human host cell a nucleic acid molecule selected from the group consisting of
   (a) a nucleic acid molecule having the nucleotide sequence shown in SEQ ID NO:1 or 3;
   (b) a nucleic acid molecule encoding a polypeptide having the amino acid sequence shown in SEQ ID NO:2 or 4;
   (c) a nucleic acid molecule encoding a fragment of a polypeptide encoded by a nucleic acid molecule of (a) or (b), wherein said fragment has cis-aconitic acid decarboxylase (CAD) activity;
   (d) a nucleic acid molecule which is at least 50% identical to a nucleic acid molecule as defined in any one of (a) to (c) and which encodes a polypeptide having CAD activity; and
   (e) a nucleic acid molecule, the complementary strand of which hybridizes under stringent conditions to a nucleic acid as defined in any one of (a) to (d) and which encodes a polypeptide having CAD activity; and
(ii) cultivating said host cell.

In a second aspect, the present invention provides a non-human host cell comprising a nucleic acid molecule which is one of the following:
(a) a nucleic acid molecule having the nucleotide sequence shown in SEQ ID NO:1 or 3;
(b) a nucleic acid molecule encoding a polypeptide having the amino acid sequence shown in SEQ ID NO:2 or 4;
(c) a nucleic acid molecule encoding a fragment of a polypeptide encoded by a nucleic acid molecule of (a) or (b), wherein said fragment has cis-aconitic acid decarboxylase (CAD) activity;
(d) a nucleic acid molecule which is at least 50% identical to a nucleic acid molecule as defined in any one of (a) to (c) and which encodes a polypeptide having CAD activity; and
(e) a nucleic acid molecule, the complementary strand of which hybridizes under stringent conditions to a nucleic acid as defined in any one of (a) to (d) and which encodes a polypeptide having CAD activity.

In preferred embodiment, this host cell further comprises itaconic acid. Use of the host cells to produce itaconic acid is also included herein.

In a third aspect, the present invention provides a composition comprising itaconic acid and a non-human host cell comprising Irg1 polypeptide or variants thereof. It also provides a composition comprising itaconic acid and the nucleic acid molecule of the present invention.

In a fourth aspect, the present invention provides and a non-human host cell comprising a nucleic acid molecule encoding Irg1 gene or variants thereof. Also, the present invention provides a composition of matter comprising itaconic acid and a non-human host cell comprising Irg1 polypeptide or variants thereof.

In a fifth aspect, the present invention provides a host cell which comprises Irg1 nucleic acid molecule or variants thereof. A host cell which comprises Irg1 polypeptide or variants thereof is also included. Furthermore, the present invention also includes the use of Irg1 nucleic acid molecule or variants thereof or Irg1 polypeptide or variants thereof to produce itaconic acid.

In a sixth aspect, the present invention provides a kit for the production of itaconic acid comprising a host cell which expresses Irg1 polypeptide or variants thereof.

### FIGURES

FIG. 1: (A) Levels of mRNA (left y-axis, black bars) or itaconic acid (right y-axis, grey bars) in resting (Ctr) or LPS-activated RAW264.7 macrophages transfected with either siRNA specific for Irg1 or with siRNA Ctr. Metabolites and RNA extractions were performed after 6 h of stimulation. The levels of Irg1 mRNA were determined by real-time RT-PCR and normalized using L27 as housekeeping gene. Each bar represents the average expression fold change (±SD). The levels of itaconic acid were determined by GC/MS measurements. Each bar represents itaconic acid levels (±SD). *p-value<0.05, **p-value<0.01. (B) Itaconic acid quantification (mM) in mouse microglial cells (BV-2 cell line) and mouse macrophages (RAW264.7 cell line) after 6 h of LPS stimulation at 10 ng/ml (grey bars). Untreated cells were used as a control (black bars). Bars represent the mean of itaconic acid concentration (±SEM). **p-value<0.01. (C and D) Levels of mRNA (C) or itaconic acid (D) in human A549 lung cancer cells transfected with the mouse Irg1 overexpressing plasmid (pmIrg1). Metabolites and RNA extractions were performed 24 h after transfection. Real-time RT-PCR results are normalized using L27 as housekeeping gene and are shown as average expression fold change (±SEM). *p-value<0.05, **p-value<0.01. (E) RAW264.7 cells were treated with LPS (10 ng/ml) at different time points (h, hours) and analyzed for Irg1 expression and itaconic acid concentration (mM).

FIG. 2: (A) Synthesis pathway of itaconic acid in the TCA cycle. Itaconic acid can only contain one labeled carbon if produced in the first round of the TCA cycle (yellow-marked atoms). (B) Labeling of citric acid (black bars) and itaconic acid (gray bars) using glucose as a tracer in RAW264.7 macrophages. The major fraction of labeled itaconate contains one isotope whereas citrate contains mainly two labeled atoms.

FIG. 3: Purification of cis-aconitate decarboxylase from HEK293T cells transfected with the pCMV6-Entry-Irg1 expression plasmid. (A) Extracts from cells transfected with empty plasmid or Flag-Irg1 plasmid were loaded onto an affinity resin (Cell MM2, FlagM purification kit, Sigma Aldrich) and proteins were eluted with Flag peptide. Cis-aconitate decarboxylase activity was measured in cell extracts and purification fractions as described in the Materials and Methods section. (B) 12 μl of each protein fraction was loaded onto an SDS-PAGE gel that was stained with Coomassie Blue. (C) Western Blot analysis of the same protein fractions was performed using an Irg1-specific antibody.

P: pellet; SN: supernatant; FT: flow through; W: wash; F1-F3: elution fractions.

FIG. 4: Human Irg1 expression and itaconic acid production. (A and B) Levels of mRNA and itaconic acid in resting (Ctr) and LPS-activated (10 μg/ml) PBMCs-derived macrophages. RNA and metabolites extractions were performed after 6 h of stimulation. (A) The levels of Irg1 mRNA were determined by real-time RT-PCR and normalized using L27 as housekeeping gene. Each bar represents the average expression fold change of three technical replicates (±SEM). (B) The levels of itaconic acid were determined by GC/MS measurements. Each bar represents itaconic acid levels (±SEM). *p-value<0.05, **p-value<0.01. (C and D) Differential Irg1 gene expression analysis and itaconic acid production between 5 different donors. (E and F) Levels of mRNA (E) or itaconic acid (F) in human A549 lung cancer cells transfected with the human Irg1 overexpressing plasmid (phIrg1). Metabolites and RNA extractions were performed 24 h after transfection. Real-time RT-PCR results are normalized using L27 as housekeeping gene and are shown as average expression fold change (±SEM). *p-value<0.05, **p-value<0.01.

FIG. 5: Mouse peritoneal macrophages from eight saline and seven LPS injected mice (1 mg/Kg) were isolated and pooled 24 h after intraperitoneal injection. (A) Irg1 expression levels and (B) itaconic acid production were analyzed compared to intraperitoneally saline-injected mice. Bars represent the mean of three technical replicates (±SEM).

FIG. 6: Effect of itaconic acid on the bacterial growth. (A) Schematic of the glyoxylate shunt. (B) GFP-expressing M. tuberculosis bacteria were cultured in 7H9 medium supplemented with acetate and various concentrations of itaconate (5, 10, 25, 50 mM). Growth was measured as relative light units (RLU) at indicated time points (d, days). Curves represent the mean of three technical replicates. (C) S. enterica was grown in liquid medium with acetate in the presence of different concentrations of itaconic acid (5, 10, 50, 100 mM). The OD was measured every hour (h). Curves are calculated in relative to time 0 and represent the mean of three independent experiments. (D) RAW264.7 cells were transfected with either siRNA specific for Irg1 (siIrg1) or with siRNA control (siNeg). After 24 hours, the cells were infected with S. enterica at a multiplicity of infection of 1:10 and incubated for 1 h at 37° C. (see Materials and Methods section). Bars represent the mean of the numbers of bacteria per ml (±SEM) obtained from three independent experiments. *p-value<0.05.

FIG. 7: Itaconic acid in mouse primary microglial cells. Primary microglial cells were treated for 6 h with LPS (1 ng/ml) (grey bars) or left untreated (black bars). Bars represent the mean of itaconic acid levels (±SD). *p-value<0.05.

FIG. 8: Multiple sequence alignment of cis-aconitic acid decarboxylase (CAD) (Aspergillus terreus), immune response gene 1 (IRG1) protein homolog (human), immune response gene 1 (Irg1) protein (mouse) and immunodisuccinate (IDS) epimerase (Agrobacterium tumefaciens). Between CAD1 and IRG1 five from eight active site residues are conserved. Conserved residues are shown in red; residues assumed to build active site are indicated with green triangles below the alignment. Figure was drawn with ESPript. Sequences were obtained from UniProt Knowledgebase (UniProtKB) with the following accession numbers: B3IUN8 (CAD1), A6NK06 (IRG1 human) P54987 (Irg1 mouse) and Q1L4E3 (IDS epimerase).

FIG. 9: Gene Tree of mouse Irg1. Gene Tree was generated using the Ensemble gene orthology/paralogy prediction method pipeline (49). The left part shows the evolutionary history of Irg1 across species. The right part shows a multiple sequence alignment of the associated proteins. Green bars shows areas of amino acid alignment, white areas are gaps in the alignment.

FIG. 10: TNF-α expression in LPS-activated human PBMCs-derived macrophages. RNA extractions were performed after 6 h of LPS (10 μg/ml) stimulation of PBMCs-derived macrophages from five different donors (D). The levels of TNF-α mRNA were determined by real-time RT-PCR and normalized using L27 as housekeeping gene. Each bar represents the average expression fold change of three technical replicates (±SEM). **p-value<0.01.

FIG. 11: (A) Levels of mRNA or (B) itaconic acid in LPS-activated RAW264.7 macrophages transfected with either siRNA specific for iNOS or with siRNA Ctr. Metabolites and RNA extractions were performed after 6 h of stimulation. The levels of iNOS mRNA were determined by real-time RT-PCR and normalized using L27 as housekeeping gene. Each bar represents the average expression fold change (±SEM) from three independent experiments. The levels of itaconic acid were determined by GC/MS measurements. Each bar represents itaconic acid levels (±SEM). **p-value<0.01.

FIG. 12: (A-C) Itaconic acid levels in resting (Ctr) and LPS-activated (10 μg/ml) PBMCs-derived macrophages from three donors treated with DEA NONOate at different concentrations (1, 10, 100 μM). Metabolites were harvested after 12 h of stimulation and the levels of itaconic acid were determined by GC/MS measurements. Each bar represents the mean of itaconic acid levels from three technical replicates (±SEM). (D) After 12 h, 180 μl of medium was harvested and combined with 20 μl of 1 mM NaOH on ice to stop the dissociation reaction. Levels of nitrite were determined using the Griess assay and the concentrations were determined against a nitrite standard curve. Bars represent the mean of nitrite concentration (μg/ml) from the three donors (±SEM).

FIG. 13: GFP-expressing M. tuberculosis bacteria were cultured in 7H9 medium supplemented with different carbon sources and various concentrations of itaconate (5, 10, 25, 50 mM) or cis-aconitate as indicated: (A) glycerol and itaconate, (B) acetate and cis-aconitate, (C) glycerol and cis-aconitate and (D) glycerol, propionate and itaconate. Growth was measured as relative light units (RLU) at indicated time points (d, days). Curves represent the mean of three technical replicates.

FIG. 14: (A) S. enterica was grown in liquid medium with glucose in the presence of itaconic acid and the optical density (OD) was measured every hour (h). Curves represent the mean of two independent experiments. (B) S. enterica was grown in liquid medium with acetate as unique carbon source in the presence of increasing concentrations of cis-aconitate (5, 10, 50 mM). The OD was measured every hour (h) to record the bacterial growth. Curves are calculated in % relative to time 0 and represent the mean of two independent experiments.

FIG. 15: (A) Levels of Irg1 mRNA or (B) itaconic acid in RAW264.7 cells transfected with either siRNA specific for Irg1 or with siRNA control under S. enterica infection at a MOI of 1:1 or 1:10 bacteria per macrophages. Infections were performed after 24 h of transfection and incubated for 0 h or 4 h after 1 h gentamycin exposure. Macrophages were then lysed to extract RNA and metabolites. Bars represent the results from one experiment.

FIG. 16: Effect of Irg1 silencing in macrophages on the bacterial growth. Mouse RAW264.7 cells were transfected with either siRNA specific for Irg1 or with siRNA specific for Aco2 as control. Macrophages were infected with S. enterica at a MOI of 1:1 or 1:10 bacteria per macrophages. Infections were performed after 24 h of transfection and incubated for 0 h or 4 h after 1 h gentamycin exposure. Bars represent the mean of the numbers of colonies (±SEM) obtained from one experiment.

FIG. 17: SEQ ID NO: 1-4. SEQ ID NO 1 relates to human Irg1 gene. SEQ ID NO 2 relates to human Irg1 polypeptide. SEQ ID NO 3 relates to mouse Irg1 gene. SEQ ID NO 4 relates to mouse Irg1 polypeptide.

FIG. 18:

Purification of IRG1 from HEK293T cells transfected with human and mouse pCMV6-Irg1 overexpression or empty pCMV6-Entry control (Ctr) plasmid. Protein extracts were loaded onto an affinity resin and eluted either by competition with FLAG peptide or by acidic conditions. Western blot analysis of protein factions eluted with FLAG peptide (A) after purification with vivaspin columns using specific IRG1 and Anti-Flag antibodies. (B) Silver staining of four human and mouse IRG1 protein fractions after elution by acidic conditions. M, marker; F1-F3, protein fractions eluted with Flag peptide; E1-E4, protein fractions eluted by acidic conditions. (Author's own work)

FIGS. 19A and B:

Michaelis-Menten enzyme kinetics for mouse and human itaconic acid production. Mouse (left side) and human (right side) IRG1 enzyme was produced in HEK293T cells transfected with pCMV6-overexpression constructs and purified with anti-FLAG resin. Itaconic acid production was measured after time periods of 5 min and 15 min of reaction. Cis-aconitic acid was used as substrate in the range of 0 to 1 mmol·l⁻¹. Michaelis-Menten constant (K_{M}, vertical dashed line) is calculated based on the rate of itaconic acid production dependent on substrate concentration. (Author's own work)

### DETAILED DESCRIPTION OF THE INVENTION

Irg1 is a gene highly expressed in mammalian macrophages during inflammation. Irg1 was originally identified as a 2.3 kb cDNA from a library synthesized from mRNA isolated from a murine macrophage cell line after lipopolysaccharide (LPS) stimulation (12).

Although the expression levels of Irg1 have been extensively studied, its cellular function has not been addressed and was unknown for a long time. Based on sequence homology. Irg1 has been classified into the MmgE/PrpD family (17), which contains some proteins for which enzymatic activities have been identified in microorganisms (18).

The inventors have surprisingly discovered that mammalian Irg1 exhibits enzymatic activity. It has been found that Irg1 has a similar function as cis-aconitic acid decarboxylase (CAD) in Aspergillus terreus, and thus can be used to catalyze the decarboxylation of cis-aconitate to itaconic acid, for example as described in the appended examples.

In fact, the present inventors demonstrated that Irg1 has cis-aconitate decarboxylase (CAD) activity both in vivo and in vitro (see the appended examples). Moreover, the present inventors showed that Irg1 having CAD-activity, being either from human or muse, has a Michaelis-Menten constant (K_{M}) that is two orders of magnitude lower than the Km of a fungal cis-aconitate decarboxylase. Thus, the use of a Irg1 sequence of the present invention instead of, e.g. a fungal CAD sequence may significantly increase itaconic acid production in a non-human host cell. Furthermore, in view of the fact that the Irg1 gene/protein of the present invention originates from mammals such as human or mouse, it is assumed that the enzyme may still be active at higher temperatures and, thus, it may be advantageous for expression in a non-human host cell, such as a fungal or yeast cell, since it may still confer a sufficient enzymatic activity to the fungus or yeast at temperatures above 30° C.

Thus, the present application provides a novel strategy for the production of itaconic acid by expressing mammalian Irg1 or a variant thereof in heterologous host cells.

Itaconic acid is an organic acid that is used in a wide range of industries. It is used at an industrial scale and large amounts of it are required. Since the achieved production rates of itaconic acid are relatively low and the overall process expensive there is a strong interest for improving the biotechnological production of itaconic acid. Innovations by which the process becomes more efficient, less expensive and energy-saving are necessary. The sequences of the present invention are believed to aid in increasing production rates of itaconic acid in host cells, preferably non-human host cells.

The present invention meets such needs, and further provides other related advantages. Using the expression of mammalian Irg1 or variants thereof in a host cell to produce itaconic acid provides an alternative or even improved approach to improve currently used industrial production of itaconic acid. A. terreus is presently the mostly frequently used commercial producer of IA, there is a need to be able to produce the acid in other microorganisms that are not as sensitive to particular fermentation conditions (e.g. substrate impurities) or which have a more favourable product composition. For example, growing filamentous fungi may cause particular problems in bioreactors, therefore, it may be more preferred to product itaconic acid in host cells that are more easily to handle. Previous attempts have been made to find better ataconic acid producing strains by mutagenesis. For example, mutated A. terreus has been shown to produce higher amount of itaconic acid. Nevertheless, this does ideally solve the issues of the sensitivity to medium components and necessity to pretreat raw materials before fermentation.

The present invention has, by using recombinant DNA technology, for the first time made it possible to obtain itaconic acid by expressing an enzyme of mammalian origin. Enzymes from different species often vary in their stability and activity. Several parameters are known to influence stability and activity of enzymes e.g. pH, temperature, concentration of respective enzymes, presence of substrate and/or product or presence of ions. Without wishing to be bound by the theory, it is believed that the present mammalian enzyme Irg1 can be heterologously expressed at in host cells which can be cultured at a wider range of temperatures than previously possible. Using host cells which have an improved temperature-tolerance will allow fermentation at a higher temperature and reduction of the cost of cooling.

The type of host cell used will also allow further improvements including, but not limited to, higher production rates, usage of alternative substrates like alternative carbon sources, alternative fermentation conditions (e.g. pH, temperature, oxygen concentration, agitation), use of alternative types of fermentors, and upscaling of production.

In a first aspect, the present invention provides a method of producing itaconic acid, comprising expressing a nucleic acid molecule encoding a Irg1 gene or a variant thereof in a host cell. Preferably, the cell is a non-human host cell such as non-mammalian host cell.

In a preferred embodiment, present invention provides a method for the production of itaconic acid, comprising
(i) expressing in a host cell, preferably non-human host cell a nucleic acid molecule selected from the group consisting of
   (a) a nucleic acid molecule having the nucleotide sequence shown in SEQ ID NO:1 or 3;
   (b) a nucleic acid molecule encoding a polypeptide having the amino acid sequence shown in SEQ ID NO:2 or 4;
   (c) a nucleic acid molecule encoding a fragment of a polypeptide encoded by a nucleic acid molecule of (a) or (b), wherein said fragment has cis-aconitic acid decarboxylase (CAD) activity;
   (d) a nucleic acid molecule which is at least 50% identical to a nucleic acid molecule as defined in any one of (a) to (c) and which encodes a polypeptide having CAD activity; and
   (e) a nucleic acid molecule, the complementary strand of which hybridizes under stringent conditions to a nucleic acid as defined in any one of (a) to (d) and which encodes a polypeptide having CAD activity; and
(ii) cultivating said host cell.

Unless otherwise indicated, the term “nucleic acid molecule” refers both to naturally and non-naturally occurring nucleic acid molecules. Non-naturally occurring nucleic acid molecules include cDNA as well as derivatives such as PNA.

The term “nucleotide sequence” or “nucleic acid molecule” refers to a polymeric form of nucleotides (i.e. polynucleotide) of at least 10 bases in length which are usually linked from one deoxyribose or ribose to another. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native internucleoside bonds, or both. The term “nucleotide sequence” does not comprise any size restrictions and also encompasses nucleotides comprising modifications, in particular modified nucleotides, e.g., as described herein.

In this regard, a nucleic acid being an expression product is preferably a RNA, whereas a nucleic acid to be introduced into a cell is preferably DNA.

The nucleic acid can be in any topological conformation. For instance, the nucleic acid can be single-stranded, double-stranded, triple-stranded, quadruplexed, partially double-stranded, branched, hairpinned, circular, or in a padlocked conformation.

The term “nucleotide sequence” includes single and double stranded forms of DNA or RNA. A nucleic acid molecule of this invention may include both sense and antisense strands of RNA (containing ribonucleotides), cDNA, genomic DNA, and synthetic forms and mixed polymers of the above. They may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.) Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

A nucleic acid molecule encoding a Irg1 gene is for example the human Irg1 gene as shown in SEQ ID NO: 1 or a mouse Irg1 gene as shown in SEQ ID NO: 3. However, it should be understood that the nucleic acid molecule is not limited to SEQ ID NO: 1 or 3.

Included in the present application are also variants Irg1 genes. As used herein a “variant” of a nucleic acid molecule encoding the Irg1 gene refers to any alteration in the wild-type gene sequence, and includes variations that occur in coding and non-coding regions, including exons, introns, promoters and untranslated regions. A “variant” of a nucleic acid molecule also refers to a nucleic acid molecule that comprises degenerate codon substitutions or combinations of deoxyribo- and ribo-nucleotides, and combinations of bases, including uracil, adenine, thymine, cytosine, guanine, inosine, xanthine hypoxanthine, isocytosine, isoguanine, etc. A “variant” of a nucleic acid molecule may also comprise a deletion or an insertion of a nucleotide. As used herein, a “variant” of a nucleic acid molecule includes a homologue of a nucleic acid molecule. A “variant” of a nucleic acid molecule encoding Irg1 further includes any nucleic acid molecule that hybridizes to a nucleic acid molecule in (a) to (d) of claim 1 under stringent conditions. A “variant” of a nucleic acid molecule also refers to the complement of any such nucleic acid sequence described above. As used herein all “variants” of a nucleic acid molecule encode a polypeptide that has CAD activity. A “variant” of a polypeptide is defined herein as a polypeptide comprising an alteration or modification(s), such as a substitution, insertion, and/or deletion, of one or more amino acid residues at one or more (several) specific positions. The altered polynucleotide is can be obtained by for instance modification of a polynucleotide sequence. The variant Irg1 proteins which the nucleotide encodes are preferably homologous to SEQ ID NO 2 or 4. A polypeptide encoded by a variant nucleic acid molecule has CAD activity. Likewise, a variant polypeptide encoded by a nucleic acid molecule of the present invention has CAD activity.

The term “CAD” is used herein as abbreviation for the fungal enzyme cis-aconitic acid decarboxylase (cis-aconitate decarboxylase), e.g. the CAD of A. terreus (as described in Dwiarti et al., J. Bioscience and Bioengineering, 94 (1):29-33, 2002). The term “cis-aconitate” refers to “cis-aconitic acid” as well as “cis-aconitic acid.” “CAD activity” refers to the ability of a polypeptide to catalyze the decarboxylation of cis-aconitate to itaconic acid.

In one aspect of the present invention, the nucleic acid molecule of the present invention includes:
(a) a nucleic acid molecule having the nucleotide sequence shown in SEQ ID NO:1 or 3;
(b) a nucleic acid molecule encoding a polypeptide having the amino acid sequence shown in SEQ ID NO:2 or 4;
(c) a nucleic acid molecule encoding a fragment of a polypeptide encoded by a nucleic acid molecule of (a) or (b), wherein said fragment has cis-aconitic acid decarboxylase (CAD) activity;
(d) a nucleic acid molecule which is at least 50% identical to a nucleic acid molecule as defined in any one of (a) to (c) and which encodes a polypeptide having CAD activity; and
(e) a nucleic acid molecule, the complementary strand of which hybridizes under stringent conditions to a nucleic acid as defined in any one of (a) to (d) and which encodes a polypeptide having CAD activity.
The first nucleic acid molecule is also referred to herein as “nucleic acid molecule (a)” or simply “(a)”. Likewise, the second nucleotide sequence nucleic acid molecule, respectively, is also referred to herein as “nucleotide sequence (b)” or simply “(b)”. The following nucleic acid molecules are named analogously and consequently refer to nucleic acid molecule (c) to (e) or simply “(c)”, “(d)” or “(e)”.

### Nucleic Acid Molecule (a)

Nucleic acid molecule (a) refers to the human immune response gene 1 (Irg1) having the nucleotide sequence shown in SEQ ID NO:1 or the mouse immune response gene 1 (Irg1) having the nucleotide sequence shown in SEQ ID NO:3.

### Nucleic Acid Molecule (b)

Nucleic acid molecule (b) refers to protein encoded by human immune response gene 1 (Irg1) having the amino acid sequence shown in SEQ ID NO:2 or the protein encoded by mouse immune response gene 1 (Irg1) having the amino acid sequence shown in SEQ ID NO:4.

### Nucleic Acid Molecule (v)

Nucleic acid molecule (c) refers to a fragment of a polypeptide encoded by a nucleic acid molecule of (a) or (b), wherein said fragment has cis-aconitic acid decarboxylase (CAD) activity.

The term “a fragment of a polypeptide encoded by a nucleic acid molecule of (a) or (b)” refers to polypeptides having one or more amino acids deleted at the N-terminus or the C-terminus of the polypeptide which is encoded by a nucleic acid molecule of (a) or (b).

In general, the term “polypeptide fragment” or “fragment” of a polypeptide as used herein refers to a polypeptide that has an amino-terminal and/or carboxy-terminal deletion compared to a full-length polypeptide. Fragments have preferably the same biological activity as the full-length polypeptide which in this case is the CAD activity.

### CAD Activity

Regarding the polypeptides encoded by nucleic acid molecule (c), (d), or (e) “having cis-aconitic acid decarboxylase (CAD) activity” means that said polypeptide catalyzes the decarboxylation of cis-aconitate to itaconic acid. CAD activity of a polypeptide of the invention encoded by a nucleic acid molecule of the present invention is preferably determined by means and methods known in the art. For example, a skilled person is able to determine the cis-aconitic acid decarboxylase (CAD) activity using methods known in the art or methods disclosed e.g. in WO/2009/014437, US20100330631 or Dwiarti et al., J. Bioscience and Bioengineering, 94 (1):29-33, 2002 (62) as well as methods disclosed in the examples and materials and methods of this invention. The methods disclosed in the examples and materials and methods herein are more preferred for determining CAD activity.

### Nucleic Acid Molecule (d)

Nucleic acid molecule (d) refers to a nucleic acid molecule which is at least 50% identical to a nucleic acid molecule as defined in any one of (a) to (c) and which encodes a polypeptide having CAD activity.

The present invention provides also for nucleotide sequences which have a percentage of identity related to the above mentioned sequences of at least 50% to 99%. Thus, for example, the percentage of identity can be at least 51%, 52%, 53%, 55%, 60%, 65%, 70%, 75%, 80%, 85%, 87%, 90%, 92%, 95%, 97%, 98% or 99%. Sequence identity on nucleotide sequences can be calculated by using the BLASTN computer program (which is publicly available, for instance through the National Center for Biotechnological Information, accessible via the internet on http://www.ncbi.nlm.nih.gov/) using the default settings of 11 for wordlength (W), 10 for expectation (E), 5 as reward score for a pair of matching residues (M), −4 as penalty score for mismatches (N) and a cutoff of 100.

### Nucleic Acid Molecule (e)

Nucleic acid molecule (e) refers to a nucleic acid molecule, the complementary strand of which hybridizes under stringent conditions to a nucleic acid as defined in any one of (a) to (d) and which encodes a polypeptide having CAD activity.

The term “which hybridizes under stringent conditions” refers to hybridization conditions that are well known to or can be established by the person skilled in the art according to conventional protocols. Appropriate stringent conditions for each sequence may be established on the basis of well-known parameters such as temperature, composition of the nucleic acid molecules, salt conditions etc.: see, for example, Sambrook et al., “Molecular Cloning, A Laboratory Manual”; CSH Press, Cold Spring Harbor, 1989 or Higgins and Hames (eds.), “Nucleic acid hybridization, a practical approach”, IRL Press, Oxford 1985 (reference 54), see in particular the chapter “Hybridization Strategy” by Britten & Davidson, 3 to 15. Typical (highly stringent) conditions comprise hybridization at 65.degree. C. in 0.5.times.SSC and 0.1% SDS or hybridization at 42.degree. C. in 50% formamide, 4.times.SSC and 0.1% SDS. [0001] [0002] Hybridization is usually followed by washing to remove unspecific signal. Washing conditions include conditions such as 65.degree. C., 0.2.times.SSC and 0.1% SDS or 2.times.SSC and 0.1% SDS or 0.3.times.SSC and 0.1% SDS at 25.degree. C.-65.degree. C.

The nucleotide sequence encoding the Irg1 protein preferably is operably linked to a promoter for control and initiation of transcription of the nucleotide sequence in a host cell as defined below. The promoter preferably is capable of causing sufficient expression of the Irg1 protein in the host cell. Expression when used herein also includes that a nucleotide sequence encoding a polypeptide of the present invention is overexpressed in a host cell, preferably non-human host cell. Overexpression can, e.g., be achieved by a strong constitutive or inducible promoter or by a strong enhancer or by introducing multiple copies such as 2, 3, 4, 5, or more copies of a nucleotide sequence of the present invention into a host cell, e.g., on a plasmid, cosmid, BAY or YAC or into the genome. Promoters useful in the nucleic acid constructs of the invention include the promoter that in nature provides for expression of the Irg1 gene. Further, both constitutive and inducible natural promoters as well as engineered promoters can be used. Promotors which drive expression of the Irg1 gene in the hosts of the invention are described below and may include e.g. promoters from glycolytic genes (e.g. from a glyceraldehyde-3-phosphate dehydrogenase gene), ribosomal protein encoding gene promoters, alcohol dehydrogenase promoters (ADH1, ADH4, and the like), promoters from genes encoding amylo- or cellulolytic enzymes (glucoamylase, TAKA-amylase and cellobiohydrolase). Other promoters, both constitutive and inducible and enhancers or upstream activating sequences are described below and/or will be known to those of skill in the art. The promoters used in the nucleic acid constructs of the present invention may be modified, if desired, to affect their control characteristics. Preferably, the promoter used in the nucleic acid construct for expression of the Irg1 gene is homologous to the host cell in which the Irg1 protein is expressed.

Depending on the host/vector system utilized, any of a number of suitable transcription and translation control elements, including constitutive and inducible promoters, transcription enhancer elements, transcription terminators, etc. may be used in a expression vector (see e.g., Bitter et al. (1987) Methods in Enzymology, 153:516-544). A promoter can be inducible. Inducible promoters are well known in the art.

Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like; an araBAD promoter; in vivo regulated promoters, such as an ssaG promoter or a related promoter (see, e.g., U.S. Patent Publication No. 20040131637), apagC promoter (Pulkkinen and Miller, J: Bacteriol., 1991: 173 (1): 86-93; Alpuche-Aranda et al., PNAS, 1992; 89(21): 10079-83), a nirB promoter (Harborn et al. (1992) Mol. Micro. 6:2805-2813), and the like (see, e.g., Dunstan et al. (1999) Infect. Immun. 67:5133-5141; McKelvie et al. (2004) Vaccine 22:3243-3255; and Chatfeld et al. (1992) Biotechnol 10:888-892); a sigma70 promoter, e.g., a consensus sigma70 promoter (see, e.g., GenBank Accession Nos. AX798980, AX798961, and AX798183); a stationary phase promoter, e.g., a dps promoter, an spy promoter, and the like; a promoter derived from the pathogenicity island SPI-2 (see, e.g., WO96/17951); an actA promoter (see, e.g., Shetron-Rama et al. (2002) Infect. Immun. 70:1087-1096); an rpsM promoter (see, e.g., Valdivia and Falkow (1996). Mol. Microbiol. 22:367-378); a tet promoter (see, e.g., Hillen, W. and Wissmann, A. (1989) In Saenger, W. and Heinemann, U. (eds), Topics in Molecular and Structural Biology, Protein-Nucleic Acid Interaction. Macmillan, London, UK, Vol. 10, pp. 143-162); an SP6 promoter (see, e.g., Melton et al. (1984; Nucl. Acids Res. 12:7035-7056); and the like. Further useful promoters for bacterial host cells include the promoter obtained from the Streptomyces coelicolor agarase gene (dagA), Bacillus subtilis levansucrase gene (sacB), Bacillus licheniformis alpha amylase (amyL), Bacillus stearothermophilus maltogenic amylase gene (amyM), Bacillus amyloliquefaciens alpha amylase gene (amyQ), Bacillus licheniformis penicillinase gene (penP), Bacillus subtilis xylA and xylB genes and prokaryotic beta-lactamase gene. These promoters are all well known in the art.

When E. coli is used as the host cell, representative E. coli promoters include, but are not limited to, the β-lactamase and lactose promoter systems (see Chang et al., Nature 275:615-624, 1978), the SP6, T3, T5, and T7 RNA polymerase promoters (Studier et al., Meth. Enzymol. 185:60-89, 1990), the lambda promoter (Elvin et al., Gene 87:123-126, 1990), the trp promoter (Nichols and Yanofsky, Meth. in Enzymology 101:155-164, 1983), and the Tac and Trc promoters (Russell et al., Gene 20:231-243, 1982).

For filamentous fungal host cells suitable promoters include promoters obtained from Aspergillus oryzae TAKA amylase, Rhizomucor miehei aspartic proteinase, Aspergillus niger or awamori glucoamylase (glaA), Rhizomucor miehei lipase and the like. In case of the host cell being Ustilago maydis, a exemplary promoter is the constitutive tef, otef promoter (Spellig et al. (1996), Mol Gen Genet 252:503-509), hsp70 promoter (Holden et al., EMBO J. 8:1927-1934. A exemplary inducible promoter is the nar1 promoter (Brachmann et al., (2001), Mol Microbiol. 42:1047-63) or the crg1 promoter (Bottin et al. (1996), Mol Gen Genet 253:342-352).

When yeast is used as the host cell, exemplary yeast promoters include 3-phosphoglycerate kinase promoter, glyceraldehyde-3-phosphate dehydrogenase (GAPDH) promoter, galactokinase (GAL1) promoter, galactoepimerase promoter, and alcohol dehydrogenase (ADH) promoter.

In yeast, a number of vectors containing constitutive or inducible promoters may be used. For a review see, Current Protocols in Molecular Biology, Vol. 2, 1988, Ed. Ausubel, et al., Greene Publish. Assoc. & Wiley Interscience, Ch. 13; Grant, et al., 1987, Expression and Secretion Vectors for Yeast, in Methods in Enzymology, Eds. Wu & Grossman, 31987, Acad. Press, N.Y., Vol. 153, pp. 516-544; Glover, 1986, DNA Cloning, Vol. II, IRL Press, Wash., D.C., Ch. 3; and Bitter, 1987, Heterologous Gene Expression in Yeast, Methods in Enzymology, Eds. Berger & Kimmel, Acad. Press, N.Y., Vol. 152, pp. 673-684; and The Molecular Biology of the Yeast Saccharomyces, 1982, Eds. Strathern et al., Cold Spring Harbor Press, Vols. I and II. A constitutive yeast promoter such as ADH or LEU2 or an inducible promoter such as GAL may be used (Cloning in Yeast, Ch. 3, R. Rothstein In: DNA Cloning Vol. H5 A Practical Approach, Ed. DM Glover, 1986, IRL Press, Wash., D.C.).

Promoters suitable for driving gene expression in other types of microorganisms are also well known in the art.

In the nucleic acid construct, the 3′-end of the nucleotide acid sequence encoding the Irg1 protein preferably is operably linked to a transcription terminator sequence. Preferably the terminator sequence is operable in a host cell of choice. In any case the choice of the terminator is not critical; it may e.g. be from any fungal gene, although terminators may sometimes work if from a non-fungal, eukaryotic, gene. The transcription termination sequence further preferably comprises a polyadenylation signal.

Optionally, a selectable marker may be present in the nucleic acid construct. As used herein, the term “marker” refers to a gene encoding a trait or a phenotype which permits the selection of, or the screening for, a host cell containing the marker. A variety of selectable marker genes are available for use in the transformation of fungi. Suitable markers include auxotrophic marker genes involved in amino acid or nucleotide metabolism, such as e.g. genes encoding ornithine-transcarbamylases (argB), orotidine-5′-decaboxylases (pyrG, URA3) or glutamine-amido-transferase indoleglycerol-phosphate-synthase phosphoribosyl-anthranilate isomerases (trpC), or involved in carbon or nitrogen metabolism, such e.g. niaD or facA, and antibiotic resistance markers such as genes providing resistance against phleomycin, bleomycin or neomycin (G418). Preferably, bidirectional selection markers are used for which both a positive and a negative genetic selection is possible. Examples of such bidirectional markers are the pyrG (URA3), facA and amdS genes. Due to their bidirectionality these markers can be deleted from transformed filamentous fungus while leaving the introduced recombinant DNA molecule in place, in order to obtain fungi that do not contain selectable markers. This essence of this MARKER GENE FREE™ transformation technology is disclosed in EP-A-0 635 574, which is herein incorporated by reference. Of these selectable markers the use of dominant and bidirectional selectable markers such as acetamidase genes like the amdS genes of A. nidulans, A. niger and P. chrysogenum is most preferred. In addition to their bidirectionality these markers provide the advantage that they are dominant selectable markers that, the use of which does not require mutant (auxotrophic) strains, but which can be used directly in wild type strains.

Embodiments of the invention may utilize an expression vector that comprises a nucleic acid molecule encoding Irg1.

Suitable exemplary vectors include, but are not limited to, viral vectors (e.g., baculovirus vectors, bacteriophage vectors, and vectors based on vaccinia virus, poliovirus, adenovirus, adeno-associated virus, SV40, herpes simplex virus, and the like), phage, plasmids, phagemids, cosmids, phosmids, bacterial artificial chromosomes (BACs), bacteriophage PI, PI-based artificial chromosomes (PACs), yeast artificial chromosomes (YACs), yeast plasmids, and any other vectors suitable for a specific host cell (e.g., E. coli or yeast).

Numerous suitable expression vectors are known to those of skill in the art, and many are commercially available. The following vectors are provided by way of example: for bacterial host cells: pQE vectors (Qiagen), pBluescript plasmids, pNH vectors, lambda-ZAP vectors (Stratagene); pTrc99a, pKK223-3, pDR540, and pRIT2T (Pharmacia); for eukaryotic host cells: pXT1, pSG5 (Stratagene), pSVK3, pBPV, pMSG, and pSVLSV40 (Pharmacia). However, any other plasmid or other vector, with or without various improvements for expression, may be used so long as it is compatible with the host cell.

Standard recombinant DNA techniques can be used to perform in vitro construction of plasmid and viral chromosomes, and transformation of such into host cells including clonal propagation.

An expression vector can contain one or more selectable marker genes to provide a phenotypic trait for selection of transformed host cells such as dihydrofolate reductase or neomycin resistance for eukaryotic cell culture, or such as tetracycline or ampicillin resistance in prokaryotic host cells such as E. coli. Generally, recombinant expression vectors will include origins of replication and selectable markers permitting transformation of the host cell, e.g., the ampicillin resistance gene of E. coli, the S. cerevisiae TRP 1 gene, etc.; and a promoter derived from a highly expressed gene to direct transcription of the biosynthetic pathway gene product-encoding sequence. Such promoters can be derived from operons encoding glycolytic enzymes such as 3-phosphoglycerate kinase (PGK), x-factor, acid phosphatase, or heat shock proteins, among others.

Optional further elements that may be present in the nucleic acid constructs of the invention include, but are not limited to, one or more leader sequences, enhancers, integration factors, and/or reporter genes, intron sequences, centromers, telomers and/or matrix attachment (MAR) sequences. The nucleic acid of the invention may further comprise a sequence for autonomous replication, such as an ARS sequence. Suitable episomal nucleic acid constructs may e.g. be based on the yeast 2μ or pKD1 (Fleer et al., 1991, Biotechnology 9: 968-975 WO98/46772). Such sequences may thus be sequences homologous to the target site for integration in the host cell's genome. The nucleic acid constructs of the invention can be provided in a manner known per se, which generally involves techniques such as restricting and linking nucleic acids/nucleic acid sequences, for which reference is made to the standard handbooks, such as Sambrook and Russel (2001) “Molecular Cloning: A Laboratory Manual (3rd edition), Cold Spring Harbor Laboratory, Cold Spring Harbor Laboratory Press F. Ausubel et al, eds.,” Green Publishing and Wiley Interscience, New York (1987).

The host cells used for culturing can be obtained using recombinant methods known in the art for providing cells with the nucleic acid molecules of the present invention. These include transformation, transconjugation, transfection or electroporation of a host cell with a suitable plasmid (also referred to as vector) comprising the nucleic acid construct of interest operationally coupled to a promoter sequence to drive expression.

It is commonly known in the art how to express a nucleotide sequence that is heterologous for a host cell. For example, the skilled artisan can apply promoters, termination sequences, transcription enhancers or the like in order to express the nucleotide sequence of interest. If applicable, the skilled artisan can adapt the codon usage to that preferred by the host cell. Means and methods for doing so are commonly known in the art. Further, the skilled artisan will then transform or transduce the host cell with the nucleotide sequence of interest. Said nucleotide sequence is advantageously in the form of a vector, yet, this is not mandatory, since also “naked” nucleotide sequences can be transformed into host cell. The nucleotide sequence of interest can be integrated into the genome of the host cell or it can be kept extrachromosomally, e.g., on free-replicating plasmids.

Transformation of host cells with the nucleic acid constructs of the invention may be carried out by methods well known in the art. Such methods are e.g. known from standard handbooks, such as Sambrook and Russel (2001) “Molecular Cloning: A Laboratory Manual (3rd edition), Cold Spring Harbor Laboratory, Cold Spring Harbor Laboratory Press F. Ausubel et al, eds., “Current protocols in molecular biology”, Green Publishing and Wiley Interscience, New York (1987). Genetic modification of fungal host cells are known from e.g. EP-A-0 635 574, WO 98/46772, WO 99/60102 and WO 00/37671

Fungal cells may be transformed by a process involving protoplast formation, transformation of the protoplasts, and regeneration of the cell wall in a manner known in the art. Procedures for transformation of Aspergillus host cells are described e.g. in EP 238 023 and Yelton et al., 1984, Proceedings of the National Academy of Sciences USA 81: 1470-1474. Suitable procedures for transformation of Aspergillus and other filamentous fungal host cells using Agrobacterium tumefaciens are described in e.g. Nat. Biotechnol. 1998 September; 16(9):839-42. Erratum in: Nat Biotechnol 1998 November; 16(11):1074. Agrobacterium tumefaciens-mediated transformation of filamentous fungi. de Groot M J, Bundock P, Hooykaas P J, Beijersbergen A G. Unilever Research Laboratory Vlaardingen, The Netherlands. Suitable methods for transforming Fusarium species are described by Malardier et al., 1989, Gene 78: 147-156 and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75:1920.

### Cultivation

The present invention comprises the step of culturing the host cell in which the nucleic acid molecule of the present invention is introduced. “Culturing”, “cultivating” or “cultivation” generally refers to growing a population of microbial cells under suitable conditions in a liquid or solid medium. In particular embodiments, culturing refers to the fermentative bioconversion of a substrate to an end-product. The term “cultivating said host cell” includes growing the host cell under conditions suitable for said host cell. Cultivating conditions for the host cells of the present invention are well known to the person skilled in the art. Conditions for the culture and production of cells, including filamentous fungi, bacterial, and yeast cells, are readily available. Cell culture media in general are set forth in Atlas and Parks, eds., 1993, The Handbook of Microbiological Media. The individual components of such media are available from commercial sources.

It might be that variations of standard conditions promote the production of itaconic acid in host cells comprising a nucleic acid molecule selected from (a) to (e). Cultivation is preferably carried out in a medium containing the substrates and nutrients required by the host cell. A skilled person is able to determine the condition required for the growth of host cell. During cultivation, itaconic acid will be produced and accumulated in the medium. By cultivation, itaconic acid is produced and accumulated at higher concentration in the medium. Conditions suitable for the host cells to produce itaconic acid can be established by the person skilled in the art, including the suitable medium components, temperature, pH, dissolved oxygen, or other parameters.

A preferred method of culturing is aerobic fermentation process. The fermentation process may also be a submerged or a solid state fermentation process.

In a solid state fermentation process (sometimes referred to as semi-solid state fermentation) the transformed host cells are fermenting on a solid medium that provides anchorage points for the fungus in the absence of any freely flowing substance. The amount of water in the solid medium can be any amount of water. For example, the solid medium could be almost dry, or it could be slushy. A person skilled in the art knows that the terms “solid state fermentation” and “semi-solid state fermentation” are interchangeable. A wide variety of solid state fermentation devices have previously been described (for review see, Larroche et al., “Special Transformation Processes Using Fungal Spores and Immobilized Cells”, Adv. Biochem. Eng. Biotech., (1997), Vol 55, pp. 179 Roussos et al., “Zymotis: A large Scale Solid State Fermenter”, Applied Biochemistry and Biotechnology, (1993), Vol. 42, pp. 37-52 Smits et al., “Solid-State Fermentation-A Mini Review, 1998), Agro-Food-Industry Hi-Tech, March/April, pp. 29-36 supra). In a submerged fermentation process on the other hand, the transformed fungal host cells are fermenting while being submerged in a liquid medium, usually in a stirred tank fermenter as are well known in the art, although also other types of fermenters such as e.g. airlift-type fermenters may also be applied (see e.g. U.S. Pat. No. 6,746,862).

Substrates present in the culture for itaconic acid may include glucose or sucrose as well as raw materials which are cheaper such as starch, molasses, hydrolysates, corn syrup, wood, beet, sugarcane molasses, corn starch, glycerol, glycine or any other carbohydrate sources known to a skilled person in the art. The substrates may be pretreated before or during fermentation.

In some embodiments, the substrate may be five-carbon (C5) sugars, six-carbon (C6) sugars, and/or oligomers of C6 and C5 sugars. Examples include, but are not limited to, glucose, fructose, sucrose, maltose, xylose, arabinose, galactose, mannose, raffinose and combinations thereof. Substrated can be derived from the hydrolysis of carbohydrate polymers such as cellulose and starch. Sources of starch include plant material (such as leaves, stems, leaves, roots and grain, particularly grains derived from but not limited to corn, wheat, barley, rice, and sorghum. Exemplary feedstocks may be obtained from alfalfa, corn stover, crop residues, debarking waste, forage grasses, forest residues, municipal solid waste, paper mill residue, pomace, scraps & spoilage (fruit & vegetable processing), sawdust, spent grains, spent hops, switchgrass, waste wood chips, wood chips.

A host cell that expresses one or more of the nucleic acid molecules of this invention could be obtained using the following example: A DNA fragment encoding a nucleic acid molecule of this invention can be obtained by polymerase chain reaction from its natural source based on its coding sequence, which can be retrieved from GenBank. The DNA fragment is then operably linked to a suitable promoter to produce an expression cassette. If desired, the coding sequences are subjected to codon optimization based on the optimal codon usage in the host microorganism. The expression cassette is then introduced into a suitable microorganism to produce the genetically modified host cell disclosed herein. Positive transformants are selected and the expression of the nucleic acid molecule of this invention is confirmed by methods known in the art, e.g., a CAD enzymatic activity analysis. The modified microorganisms are then cultured in a suitable medium. Preferably, the medium contains a precursor for making itaconic acid. After a sufficient culturing period itaconic acid is isolated.

In a preferred embodiment the present invention relates to a method for the production of itaconic acid by expressing nucleic acid molecule (a), (b), (c), (d) or (e) in a heterologous host cell.

The term “heterologous” refers to what is not normally found in the host cell in nature. The term “heterologous host cell” refers to a cell other than the organism where the nucleic acid encoding the Irg1 is obtained or derived from.

### Host Cells

The host cell may be a prokaryotic cell, a yeast cell or a fungal cell, or other host cells which are commonly used for bio-fermentation. The prokaryotic cell can be a gram-negative or gram-positive. The host cell may be gram-negative prokaryotic cell like E. coli. or gram-negative prokaryotic cell like B. subtilits or B. megaterium.

Examples of host cells include microorganisms belonging to the genus Escherichia, Corynebacterium, Brevibacterium, Bacillus, Microbacterium, Serratia, Pseudomonas, Agrobacterium, Alicyclobacillus, Anabaena, Anacystis, Arthrobacter, Azobacter, Chromatium, Erwinia, Methylobacterium, Phormidium, Rhodobacter, Rhodopseudomonas, Rhodospirillum, Scenedesmus, Streptomyces, Synnechococcus, or Zymomonas.

Specific examples thereof include Escherichia coli, Bacillus subtilis, Brevibacterium immariophilum, Brevibacterium saccharolyticum, Brevibacterium flavum, Brevibacterium lactofermentum, Corynebacterium glutamicum, Corynebacterium acetoacidophilum, Microbacterium ammoniaphilum, Serratia marcescens, Agrobacterium rhizogenes, Arthrobacter aurescens, Arthrobacter nicotianae, Arthrobacter sulfureus, Arthrobacter ureafaciens, Erwinia carotovora, Erwinia herbicola, Methylobacterium extorquens, Phormidium sp., Rhodobacter sphaeroides, Rhodospirillum rubrum, Streptomyces aureofaciens, Streptomyces griseus, and Zymomonas mobilis.

Other examples include Escherichia coli XL1-Blue (manufactured by Stratagene), Escherichia coli XL2-Blue (manufactured by Stratagene), Escherichia coli DH1 (Molecular Cloning, Vol. 2, p. 505), Escherichia coli DH5a (manufactured by Toyobo Co., Ltd.), Escherichia coli MC1000 [Mol. Biol., 138 179-207 (1980)], Escherichia coli W1485 (ATCC12435), Escherichia coli JM109 (manufactured by Stratagene), Escherichia coli HB101 (manufactured by Toyobo Co., Ltd.), Escherichia coli W3110 (ATCC14948), Escherichia coli NM522 (manufactured by Stratagene), Bacillus subtilis ATCC33712, Bacillus sp. FERM BP-6030, Brevibacterium immariophilum ATCC14068, Brevibacterium saccharolyticum ATCC14066, Brevibacterium flavum ATCC14067, Brevibacterium lactofermentum ATCC13869, Corynebacterium glutamicum ATCC13032, Corynebacterium glutamicum ATCC14297, Corynebacterium acetoacidophilum ATCC13870, Microbacterium ammoniaphilum ATCC15354, Serratia marcescens ATCC13880, Agrobacterium rhizogenes ATCC11325, Arthrobacter aurescens ATCC13344, Arthrobacter nicotianae ATCC15236, Arthrobacter sulfureus ATCC19098, Arthrobacter ureafaciens ATCC7562, Erwinia carotovora ATCC15390, Erwinia herbicola ATCC21434, Methylobacterium extorquens DSM1337, Phormidium sp. ATCC29409, Rhodobacter sphaeroides ATCC21286, Rhodospirillum rubrum ATCC11170, Streptomyces aureofaciens ATCC10762, Streptomyces griseus ATCC10137, and Zymomonas mobilis ATCC10988.

“Fungi” are herein defined as eukaryotic microorganisms and include all species of the subdivision Eumycotina (Alexopoulos, C. J., 1962, In: Introductory Mycology, John Wiley & Sons, Inc., New York defined as eukaryotic microorganisms that include all filamentous forms of the subdivision Eumycotina. These fungi are characterized by a vegetative mycelium composed of chitin, cellulose, and other complex polysaccharides. The filamentous fungi used in the present invention are morphologically, physiologically, and genetically distinct from yeasts. Vegetative growth by filamentous fungi is by hyphal elongation and carbon catabolism of most filamentous fungi are obligately aerobic. “Yeasts” are herein defined as eukaryotic microorganisms and include all species of the subdivision Eumycotina that predominantly grow in unicellular form. Yeasts may either grow by budding of a unicellular thallus or may grow by fission of the organism. A fungal host cell is preferably a host cell selected from filamentous fungi.

Examples of fungal cells include Aspergillus sp., Yarrowia lipolytica, Ustilago maydis, Ustilago zeae, Candida sp., Rhodotorula sp., Pseudozyma Antarctica, including Aspergillus terreus, Aspergillus niger, Aspergillus itaconicus, and Aspergillus flavus.

A host cell as described herein further expresses or over-expresses, apart from a nucleic acid molecule of the present invention, in one embodiment one more nucleic acid molecules whose expression product contributes to an increase in the production rate of itaconic acid. Such nucleic acid molecules are described in EP2262827, EP2183367 and/or EP2017344 and encode, e.g., a malate-citrate antiporter or a mitochondrial carrier protein.

In a further embodiment the present invention relates to a method wherein the host cell used in said method is a cell which is optimized for the production of itaconic acid, such as an fungal cell optimized for batch fermentation. The ability to improve yields of itaconic acid production in host cells may be achieved by: 1) improving bioreactor performance via culturing conditions and/or media optimization; 2) improved vector expression by incorporating highly active promoters or increasing vector copy number by amplification; and/or 3) cell host optimization by enhancing endogenous pathways within the host cell line that provide for better titer yields and improved cell growth in large scale bioreactors. Any of these improvements or combinations thereof can result in processes that will shorten the number of manufacturing runs required to produce annual product needs, thereby relieving overall manufacturing constraints within the marketplace. Cell host optimization can be achieved by manipulating endogenous pathways, including mRNA transcription and maturation, protein synthesis and post-translation modifications, protein secretion and cellular sub-localization, protein trafficking between cytosol and organelles, and cell cycle and survival regulation.

Fermentation processes for growing cells is well developed and known by people skilled in the art. The fermentation process development includes medium optimization and fermentation process control parameters, optimization to achieve optimum cell growth. As used herein, the term “optimization” refers to the modification nucleic acid or the host cell as well as any treatment of said host cell which results in an increased or more cost-effective production of itaconic acid. For instance it is was reported that itaconic acid production is suppressed during cultivation since the growth of Aspergillus terreus is inhibited by the produced itaconic acid (Kobayashi et al., J. Ferment. Technol., 44, 264-274; 1966). To overcome such a product inhibition in the cultivation of a microorganism, it is preferable to select an itaconic acid-resistant mutant strain which will lead to improvement of production with high yield. Such a high itaconic acid yielding strain is e.g. the Aspergillus terreus Mutant TN-484 (60). Another example is the enhanced itaconic acid production of Aspergillus terreus SKR10 by ultraviolet, chemical and mixed mutagenic treatments (61).

In a further embodiment the present invention relates to a method wherein the host cell used in said method is a fungal cell that is selected from Aspergillus terrus MJL05 strain, Aspergillus terreus TN484, Aspergillus terreus TN484-M1, Aspergillus terreus NRRL 1960, Aspergillus terreus NRRL 1963, Aspergillus terreus NRRL 265, Aspergillus terreus DSM 23081, Aspergillus terreus LU02b, Aspergillus terreus IMI 282743, Aspergillus terreus IFO 6365 or Aspergillus terreus SKR10.

In a further embodiment the present invention relates to a method wherein the host cell used in said method is a yeast cell that is selected from Saccharomyces cerevisiae, Schizosaccharomyces pombe, Kluyveromyces lactis, Hansenula polymorpha or Pichia pastoris.

In a further embodiment the present invention relates to a method wherein the host cell is modified for industrial application, such as in scale-up production in large fermenters.

The term “modified” refers herein to modifications of said host cell that are manipulated through genetic or metabolic engineering. Strategies for the improvement of microbial strains for the overproduction of industrial products are known in the art and are for example reviewed in 58, 59.

In a further embodiment the present invention relates to a method wherein the host cell used in said method is optimized for the production of itaconic acid.

### Isolation and Further Processing

In a preferred embodiment, the present method further includes the step of isolating the itaconic acid from said host cell and/or the extracellular medium to obtain itaconic acid. Depending on the selection of host cell, itaconic acid can be isolated from said host cell after cell disrupture. Isolating can also be carried out by collecting the culture medium.

According to one preferred embodiment the present application, itaconic acid can be obtained by removing the cells and other suspended solids by filtering the cell culture broth. The filtrate can be further concentrated and the itaconic acid contained therein can thus be crystallized. and thereby obtaining itaconic acid. The term “obtained itaconic acid” refers herein to any product of said method consisting isolated, enriched or cleaned-up itaconic acid. The term “further processed” refers herein to any transfer of said product into another product including forming a solution, suspension, dispersion or mixture of the obtained itaconic acid with at least one other compound.

In a further embodiment the present method comprises further processing the itaconic acid obtained. The term “processed” refers herein to any chemical processing of itaconic acid like derivatization or polymerization, or down-stream processing like crystallization, separation, decolorization, recrystallization, drying or packing.

Itaconic acid separation is known. Host cells and solids are removed by filtration, and after evaporation at sufficiently acidic conditions, cooling and crystallisation, an industrial grade itaconic acid (e.g. for esterification) is obtained. For higher grade itaconic acid, the hot evaporate is treated with activated carbon and filtered. Mother liquor from crystallisation may then be solvent-extracted or treated by anion exchange. Recrystallisation from water gives a pure product when the substrates are glucose or sucrose. Precipitation of insoluble itaconic acid salts is also possible. Itaconic acid is then redissolved with the addition of alkali salts like ammonia.

### Composition

In a further aspect, the present invention provides a composition of matter comprising itaconic acid and a non-human host cell which comprises Irg1 polypeptide or variants thereof. It also provides a composition comprising itaconic acid and the nucleic acid molecule of the present invention. In a further embodiment the composition of matter comprises at least 1 g/l, such as at least 2, 3, 4, 5, 6, 7, 8, 9, 10, 11, 12, 13, 14, 15, 16, 17, 18, 19 or 20 g/l, itaconic acid.

In another aspect the present invention provides a non-human host cell comprising a nucleic acid molecule or a polypeptide encoded by a nucleic acid selected from any of the group comprising
(a) a nucleic acid molecule having the nucleotide sequence shown in SEQ ID NO:1 or 3;
(b) a nucleic acid molecule encoding a polypeptide having the amino acid sequence shown in SEQ ID NO:2 or 4;
(c) a nucleic acid molecule encoding a fragment of a polypeptide encoded by a nucleic acid molecule of (a) or (b), wherein said fragment has cis-aconitic acid decarboxylase (CAD) activity;
(d) a nucleic acid molecule which is at least 50% identical to a nucleic acid molecule as defined in any one of (a) to (c) and which encodes a polypeptide having CAD activity; and
(e) a nucleic acid molecule, the complementary strand of which hybridizes under stringent conditions to a nucleic acid as defined in any one of (a) to (d) and which encodes a polypeptide having CAD activity.

### Kit

In another aspect the present invention provides a kit for the production of itaconic acid comprising one of the nucleic acid molecule (a), (b), (c), (d) or (e) or a non-human host cell comprising the nucleic acid molecule.

The kit can further include the necessary components for the culture, including the host cells comprising the nucleic acid molecules and nutrients. For example, the components to form the culture may be conveniently pre-packaged in the required amounts to facilitate use in laboratory or industrial settings, without limitation. Such kit may also include labels, indicia and directions to facilitate the use of each component and the manner of combining the components in accordance with various embodiments of the present invention.

It must be noted that as used herein, the singular forms “a”, “an”, and “the”, include plural references unless the context clearly indicates otherwise.

It must be noted that as used herein, the term “Irg1” may refer to the gene Irg1 or the protein encoded by said gene. The terms “Irg1”, “Irg1 gene”, “Irg1 nucleic acid molecule” are used interchangeably herein.

The terms “Irg1”, “Irg1 protein”, “Irg1 polypeptide” might be used interchangeably herein. The Irg1 protein is also sometimes called herein “immune-responsive gene 1 protein”.

When using the abbreviation “Irg1”, the skilled person, on the basis of the context, knows when an Irg1 gene or nucleotide sequence or an Irg1 protein or amino acid sequence, respectively, of the present invention is meant. Thus, the term “Irg1” when used herein encompasses a nucleotide sequence or amino acid sequence of a protein (can be used interchangeably with the term “polypeptide”) as described herein that has preferably CAD activity.

The nucleotide sequences of the invention are preferably “isolated” or “substantially pure”. An “isolated” or “substantially pure” nucleotide sequence or nucleic acid (e.g., a RNA, DNA or a mixed polymer) is one which is substantially separated from other cellular components that naturally accompany the native polynucleotide in its natural host cell, e.g., ribosomes, polymerases, and genomic sequences with which it is naturally associated. The term embraces a nucleotide sequence or nucleic acid that (1) has been removed from its naturally occurring environment, (2) is not associated with all or a portion of a polynucleotide in which the “isolated nucleotide sequence” is found in nature, (3) is operatively linked to a polynucleotide which it is not linked to in nature, or (4) does not occur in nature. The term “isolated” or “substantially pure” also can be used in reference to recombinant or cloned DNA isolates, chemically synthesized polynucleotide analogs, or polynucleotide analogs that are biologically synthesized by heterologous systems.

However, “isolated” does not necessarily require that the nucleotide sequence or nucleic acid so described has itself been physically removed from its native environment. For instance, an endogenous nucleotide sequence in the genome of an organism is deemed “isolated” herein if a heterologous sequence (i.e., a sequence that is not naturally adjacent to this endogenous nucleic acid sequence) is placed adjacent to the endogenous nucleic acid sequence, such that the expression of this endogenous nucleic acid sequence is altered. By way of example, a non-native promoter sequence can be substituted (e.g., by homologous recombination) for the native promoter of a gene in the genome of a human cell, such that this gene has an altered expression pattern. This gene would now become “isolated” because it is separated from at least some of the sequences that naturally flank it.

A nucleotide sequence is also considered “isolated” if it contains any modifications that do not naturally occur to the corresponding nucleic acid in a genome. For instance, an endogenous coding sequence is considered “isolated” if it contains an insertion, deletion or a point mutation introduced artificially, e.g., by human intervention. An “isolated nucleotide sequence” includes a nucleic acid integrated into a host cell chromosome at a heterologous site, a nucleic acid construct present as an episome. Moreover, an “isolated nucleotide sequence” can be substantially free of other cellular material, or substantially free of culture medium when produced by recombinant techniques, or substantially free of chemical precursors or other chemicals when chemically synthesized.

A “polypeptide” refers to a molecule comprising a polymer of amino acids linked together by a peptide bond(s). Said term is herein interchangeably used with the term “protein”. A “polypeptide” encompasses both naturally-occurring and non-naturally-occurring proteins, and fragments, mutants, derivatives and analogs thereof. Polypeptides include polypeptides and peptides of any length, including proteins (for example, having more than 50 amino acids) and peptides (for example, having 2-10, 2-20, 2-30, 2-40 or 2-49 amino acids). Polypeptides include proteins and/or peptides of any activity or bioactivity. A “peptide” encompasses analogs and mimetics that mimic structural and thus biological function.

Polypeptides may further form dimers, trimers and higher oligomers, i.e. consisting of more than one polypeptide molecule. Polypeptide molecules forming such dimers, trimers etc. may be identical or non-identical. The corresponding higher order structures are, consequently, termed homo- or heterodimers, homo- or heterotrimers etc. The terms “polypeptide” and “protein” also refer to naturally or non-naturally modified polypeptides/proteins wherein the modification is effected e.g. by glycosylation, acetylation, phosphorylation and the like. Such modifications are well known in the art.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The methods and techniques of the present invention are generally performed according to conventional methods well known in the art. Generally, nomenclatures used in connection with, and techniques of biochemistry, enzymology, molecular and cellular biology, microbiology, genetics and protein and nucleic acid chemistry and hybridization described herein are those well-known and commonly used in the art.

All documents cited in this disclosure are incorporated by reference in their entirety. To the extent the material incorporated by reference contradicts or is inconsistent with this specification, the specification will supersede any such material.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the terms “including”, “containing” or sometimes when used herein with the term “having”.

As used herein the term “murine” is used interchangeably with the term “mouse”.

Without further elaboration, it is believed that one skilled in the art can, based on the above description, utilize the present invention to its fullest extent. The following specific embodiments are to be construed as illustrative, and not limitative of the remainder of the disclosure in any way whatsoever.

### EXAMPLES

### Example 1

### Irg1 Function

To study the Irg1's enzymatic function, the inventors analyzed the metabolomics profile of siRNA mediated Irg1 silencing under Lipopolysaccharide (LPS) stimulation in RAW264.7 cells (murine macrophages). It was observed that the metabolite most significantly affected by Irg1 silencing was itaconic acid. To further study the metabolic activity of Irg1, the inventors expressed murine Irg1 in A549 human lung cancer cells. It was found that cells contained high amounts of both Irg1 gene transcript and itaconic acid metabolite 24 h after transfection, but not in non-transfected cells or in cells transfected with an empty control plasmid.

Next the inventors characterized the role of Irg1 in the itaconic acid metabolic pathway. Murine Irg1 shows a 23% amino acid sequence identity to CAD expressed by the fungus Aspergillus terreus (FIG. 8). Additionally, the stable-isotope labeling experiments showed that Irg1 encodes a mammalian enzyme that catalyze the decarboxylation of cis-aconitate to itaconic acid.

The inventors purified FLAG-tagged Irg1 protein from HEK293T cells transfected with a pCMV6-Entry-Irg1 expression plasmid and showed that protein extracts prepared from those cells catalyzed the conversion of cis-aconitate to itaconic acid, while no itaconic acid formation was detected when extracts were prepared from cells transfected with an empty vector.

The inventors transfected A549 human lung cancer cells with a pCMV6 plasmid expressing human Irg1 cDNA (phIrg1) to show CAD activity of human Irg1. The inventors observed high amounts of both Irg1 gene transcript and itaconic acid 24 h post transfection.

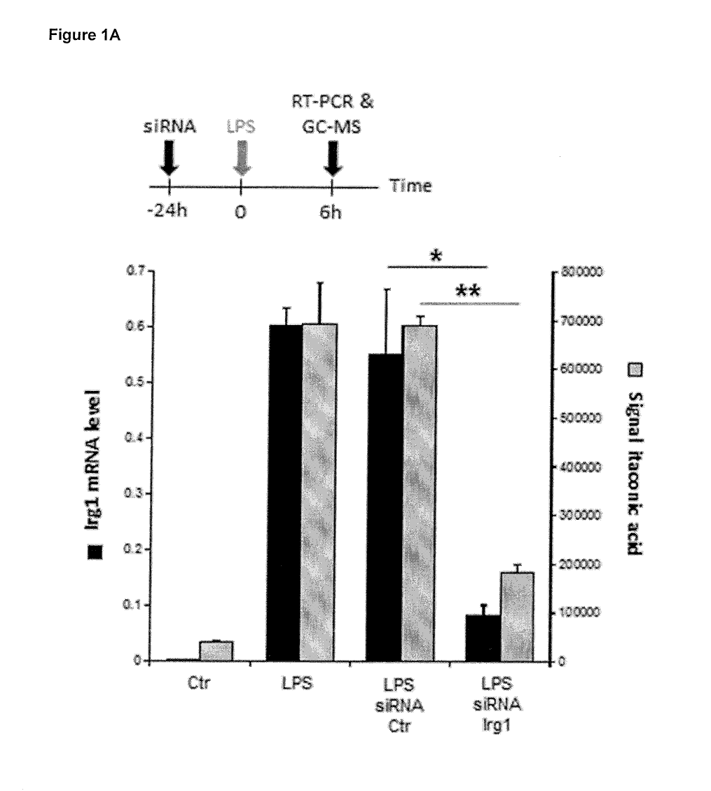

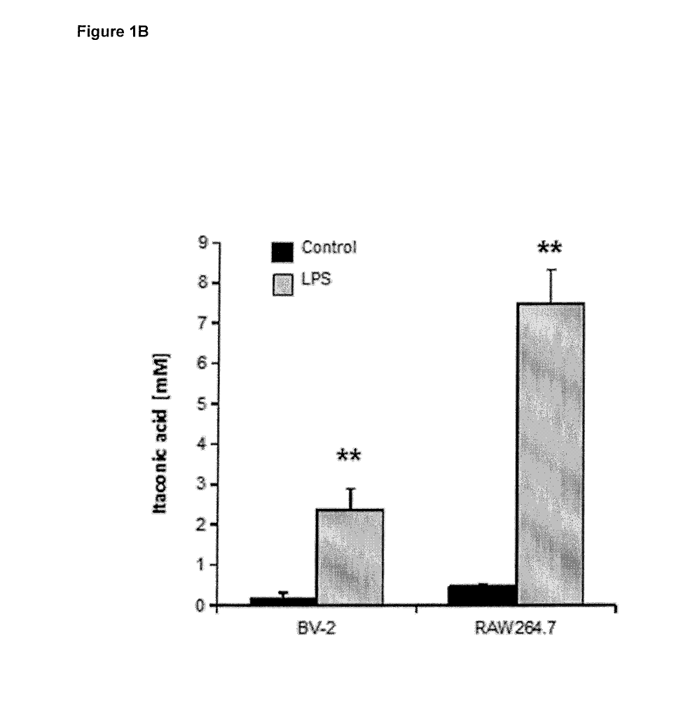

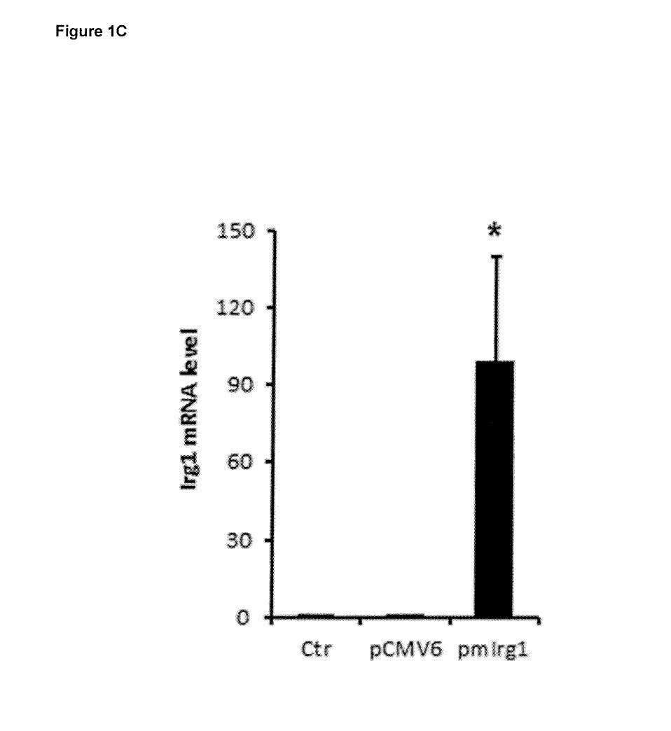

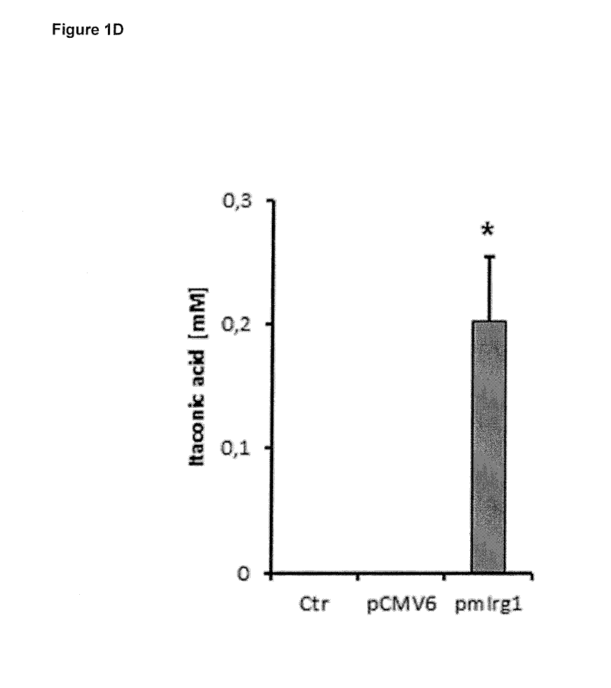

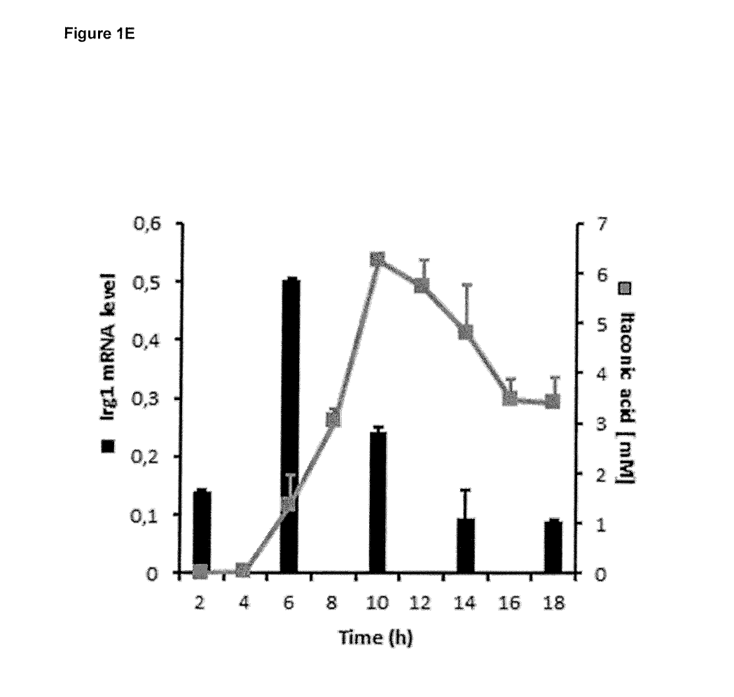

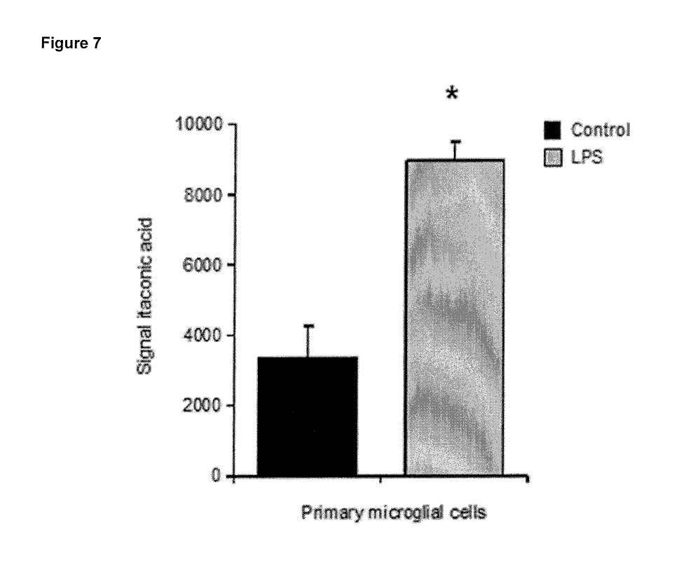

The inventors analyzed the metabolomics profile of siRNA mediated Irg1 silencing under Lipopolysaccharide (LPS) stimulation in RAW264.7 cells (murine macrophages). The inventors first confirmed that the silencing of Irg1 resulted in an 80% decrease of Irg1 mRNA level compared to non-specific siRNA control (FIG. 1A). In non-activated macrophages very low levels of Irg1 mRNA (17-fold less when compared to LPS activated cells) were detected. After silencing of Irg1 in RAW264.7 cells, the measured a total of 260 intracellular metabolites and, out of these, they found that 5 were significantly different compared to untreated RAW264.7 cells (Welch's t-test, p<0.05). Most strikingly, the inventors observed that the metabolite most significantly affected by Irg1 silencing was itaconic acid (p=2.5×10-8). Based on the experiments of the inventors/these experiments, the silencing of Irg1 elicited a 60% decrease of itaconic acid compared to control conditions (FIG. 1A). Only low levels of the metabolite (11.5-fold less compared to LPS activated cells) were measured in resting macrophages. Having identified itaconic acid as the main affected metabolite by Irg1 silencing, the inventors performed an intracellular quantification of this compound and found a concentration of 3 mM in BV-2 mouse microglial cells and 8 mM in RAW264.7 mouse macrophages after LPS treatment (LPS 10 ng/ml) (FIG. 1B). The inventors measured similar amounts in murine primary microglial cells induced by LPS treatment (FIG. 7). Such high intracellular itaconic acid concentrations after LPS treatment clearly point towards an immunological function of this metabolite.

To further study the metabolic activity of Irg1, the inventors overexpressed murine Irg1 in A549 human lung cancer cells. The inventors found that cells contained high amounts of both Irg1 gene transcript and itaconic acid metabolite (0.2±0.05 mM) 24 h after transfection, but not in non-transfected cells or in cells transfected with an empty control plasmid, where Irg1 mRNA and itaconic acid were below detection limit (FIGS. 1C and 1D).

Finally, to investigate the dynamics of Irg1 expression and itaconic acid production after a pro-inflammatory stimulus, the inventors analyzed RAW264.7 cells activated with LPS (10 ng/ml) at different time points. While Irg1 transcript was already produced after 2 h, significant amounts of itaconic acid could be measured starting 6 h after LPS treatment (FIG. 1E). The time-course of Irg1 expression is in-line with observed expression profiles of other pro-inflammatory cytokines. The positive time-dependent correlation between Irg1 expression and itaconic acid levels confirms the cellular role of Irg1 in itaconic acid production.

### Example 2

### Itaconic Acid Metabolic Pathway

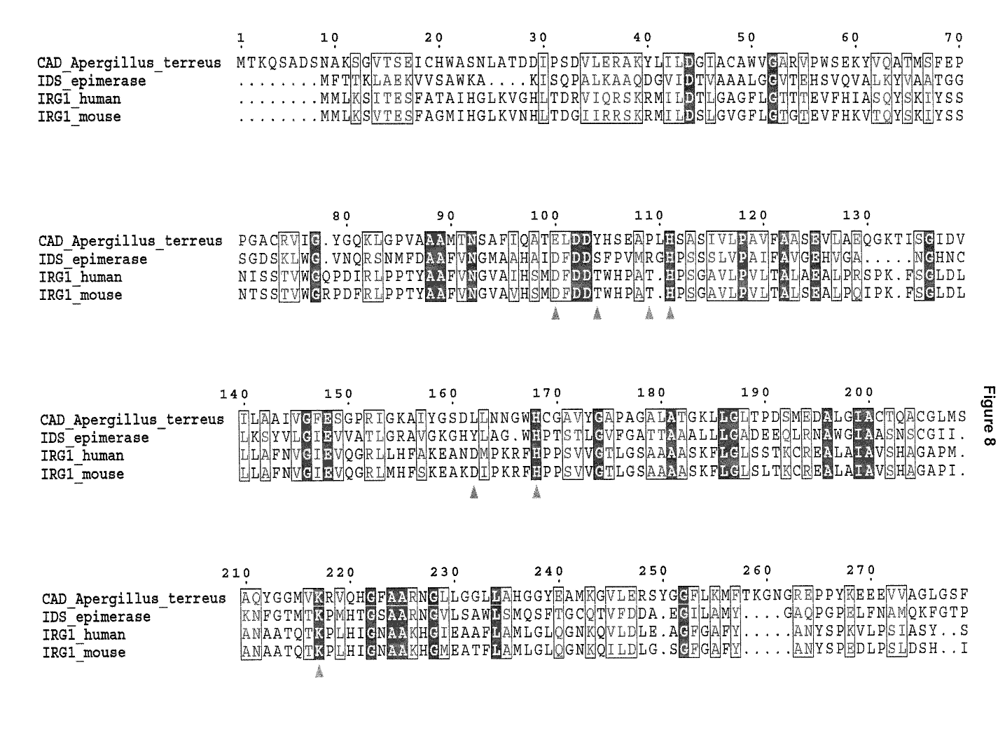

Intriguingly, murine Irg1 shows a 23% amino acid sequence identity to the enzyme cis-aconitate decarboxylase (CAD) expressed by the fungus Aspergillus terreus (FIG. 8). In fact, Irg1 is evolutionary conserved across a large set of species (FIG. 9). This fungus is commonly used for the biotechnological production of itaconic acid at industrial scale (20). The biosynthesis of this dicarboxylic acid has been of interest since it can be used as a starting material for chemical synthesis of polymers (21). The fungal CAD enzyme catalyzes the formation of itaconic acid by decarboxylating cis-aconitate to itaconic acid (22). To determine if mammalian Irg1 has a similar function as CAD in A. terreus, the inventors performed stable-isotope labeling experiments. They incubated LPS-activated RAW264.7 macrophages with uniformly 13C-labeled glucose (U-13C6). Citrate synthase catalyzes the transfer of two labeled carbon atoms from acetyl-CoA to oxaloacetate resulting in M2 cis-aconitate isotopologues (FIG. 2A). If the decarboxylation is performed by a CAD homologue, the first carbon atom of the molecule is expected to be removed during the decarboxylation resulting in M1 isotopologues of itaconic acid. The inventors determined 45% of the citrate molecules as M2 isotopologues whereas 38% of the itaconic acid molecules were M1 isotopologues (FIG. 2B). The inventors also found a significant fraction of M2, M3 and M4 itaconic acid isotopologues. The M4 fraction of itaconic acid reflects pyruvate carboxylase or reverse malic enzyme activity. Due to the symmetry of succinate, subsequent turns of the TCA cycle can result in M2 or M3 isotopologues of itaconic acid.

The observations described suggest that Irg1 encodes a mammalian enzyme that catalyzes the decarboxylation of cis-aconitate to itaconic acid.

### Example 3

### Irg1 Protein Purification and CAD Activity Assay

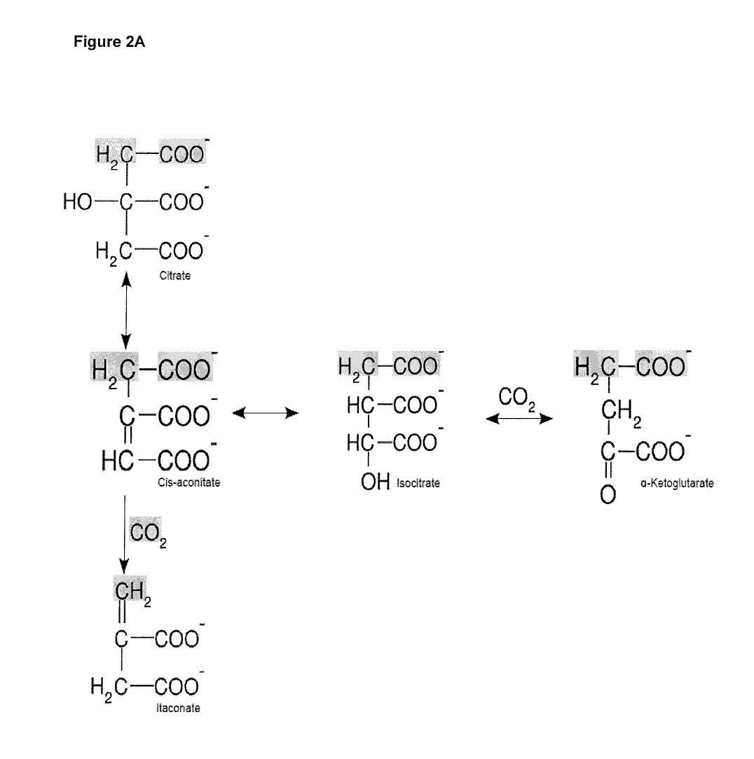

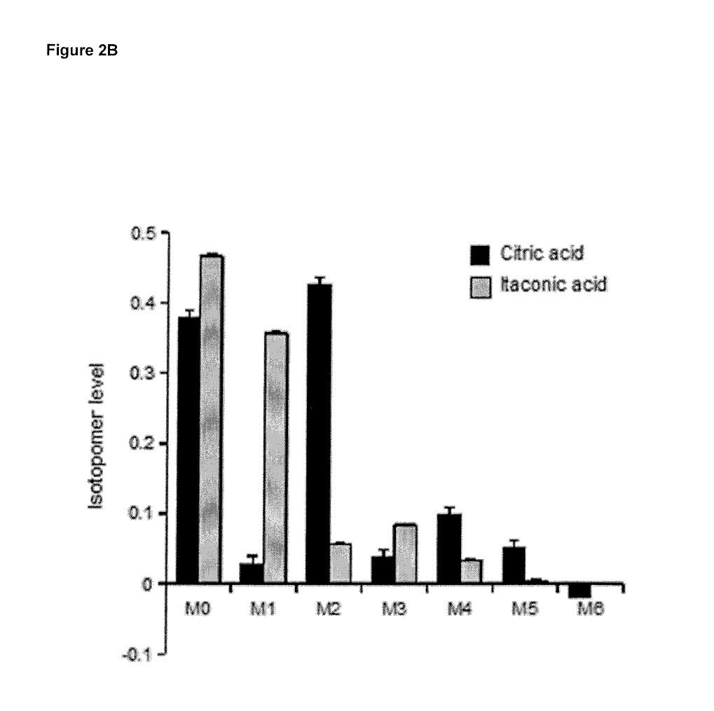

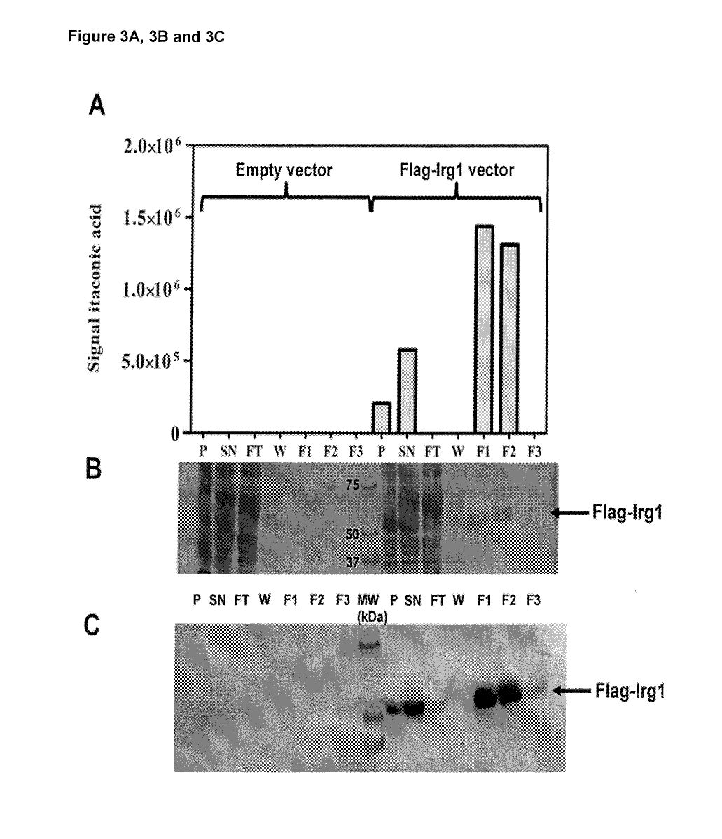

To directly demonstrate that the Irg1 protein catalyzes the decarboxylation of cis-aconitate, the inventors purified FLAG-tagged Irg1 protein from HEK293T cells transfected with a pCMV6-Entry-Irg1 expression plasmid. As depicted in FIG. 3A, protein extracts prepared from those cells catalyzed the conversion of cis-aconitate to itaconic acid. No itaconic acid formation was detected when extracts were prepared from cells transfected with an empty vector. Furthermore, affinity purification of the extract prepared from FLAG-Irg1 overexpressing cells clearly showed coelution of the cis-aconitate decarboxylase activity with a protein band identified as Irg-1 by SDS-PAGE (expected MW ˜55 kDa for Flag-Irg1; FIG. 3B) and Western blot analysis using anti-Irg1 antibody (FIG. 3C). SDS-PAGE analysis showed that this purification procedure yielded a homogenous preparation of the Irg1 protein (FIG. 3B) thus demonstrating that the cis-aconitate decarboxylase activity measured in the purified fractions was not due to another contaminating protein.

### Example 4

### Itaconic Acid is Produced by Human Primary Macrophages, but at Lower Levels Compared to Mouse Cells

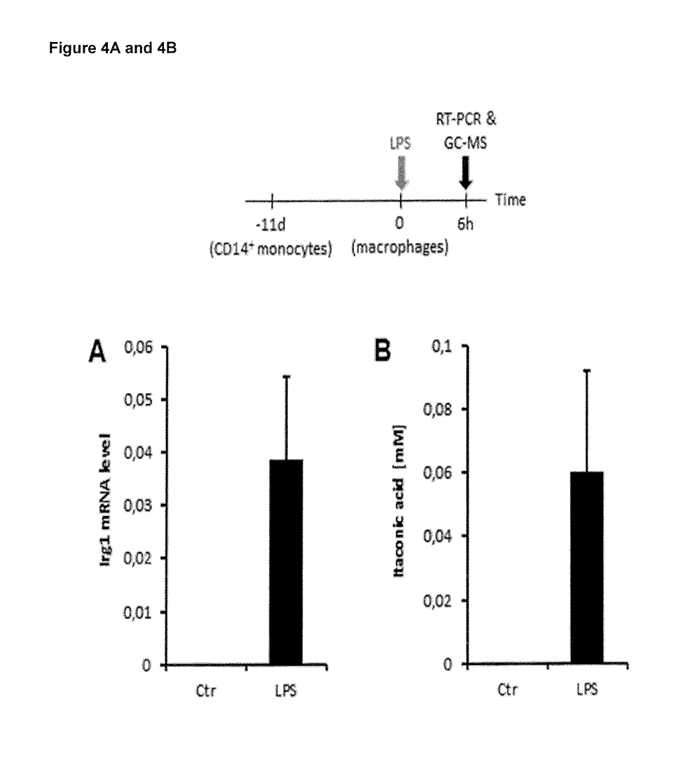

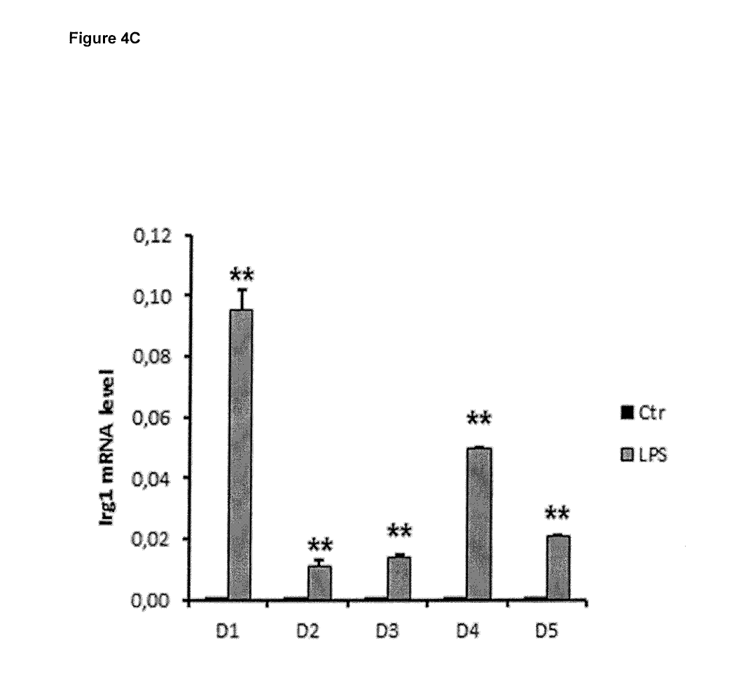

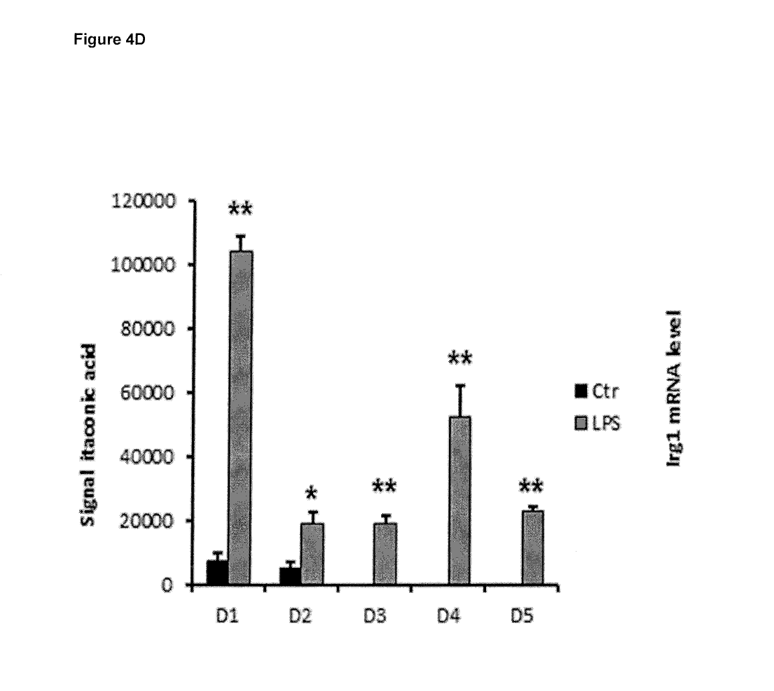

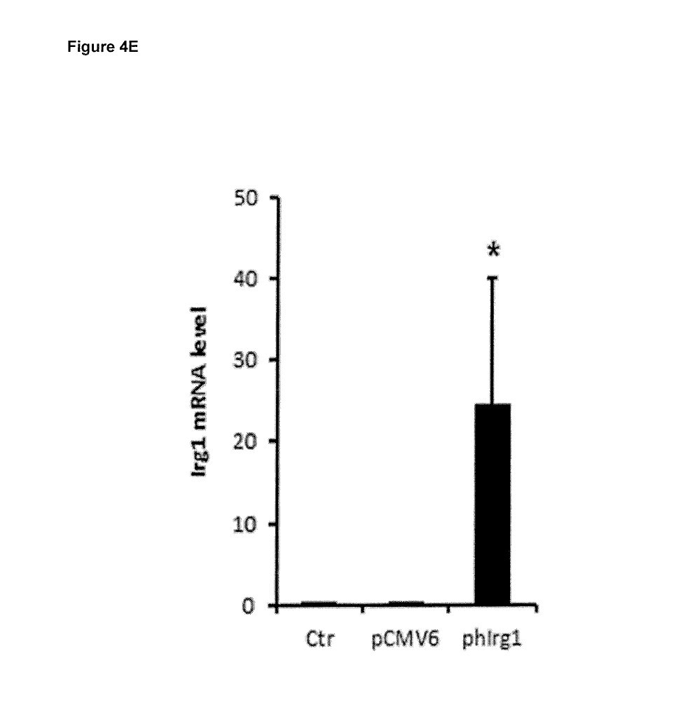

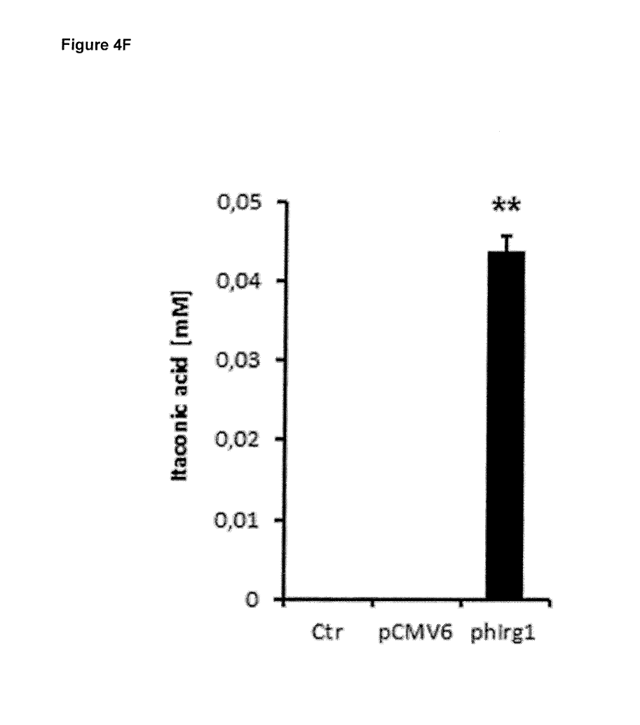

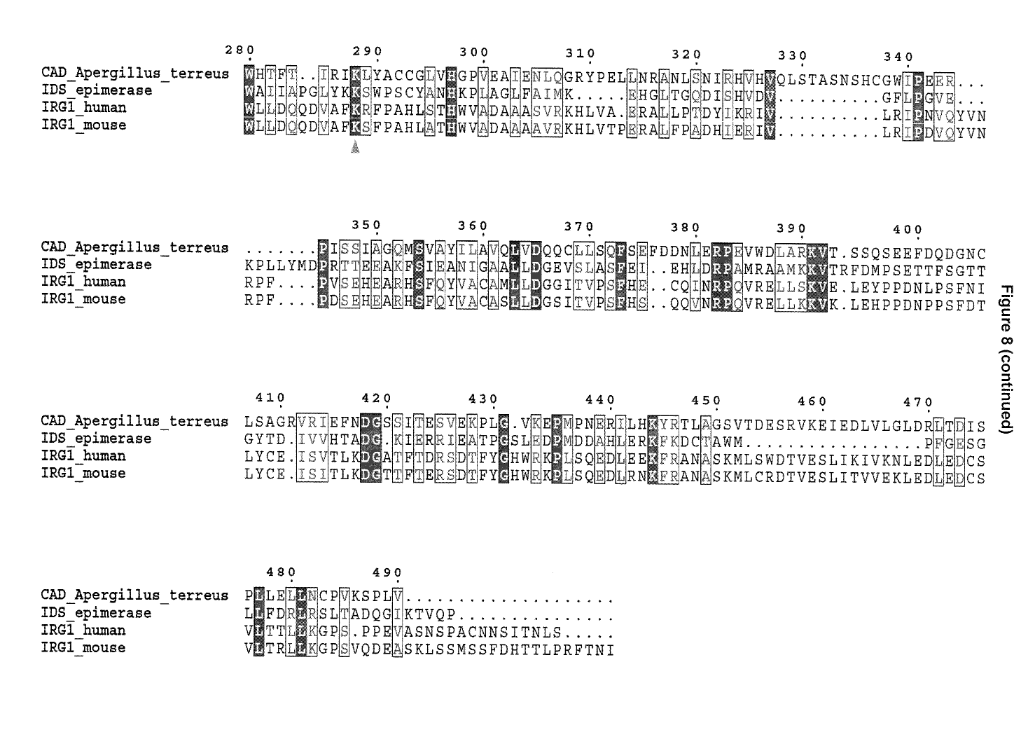

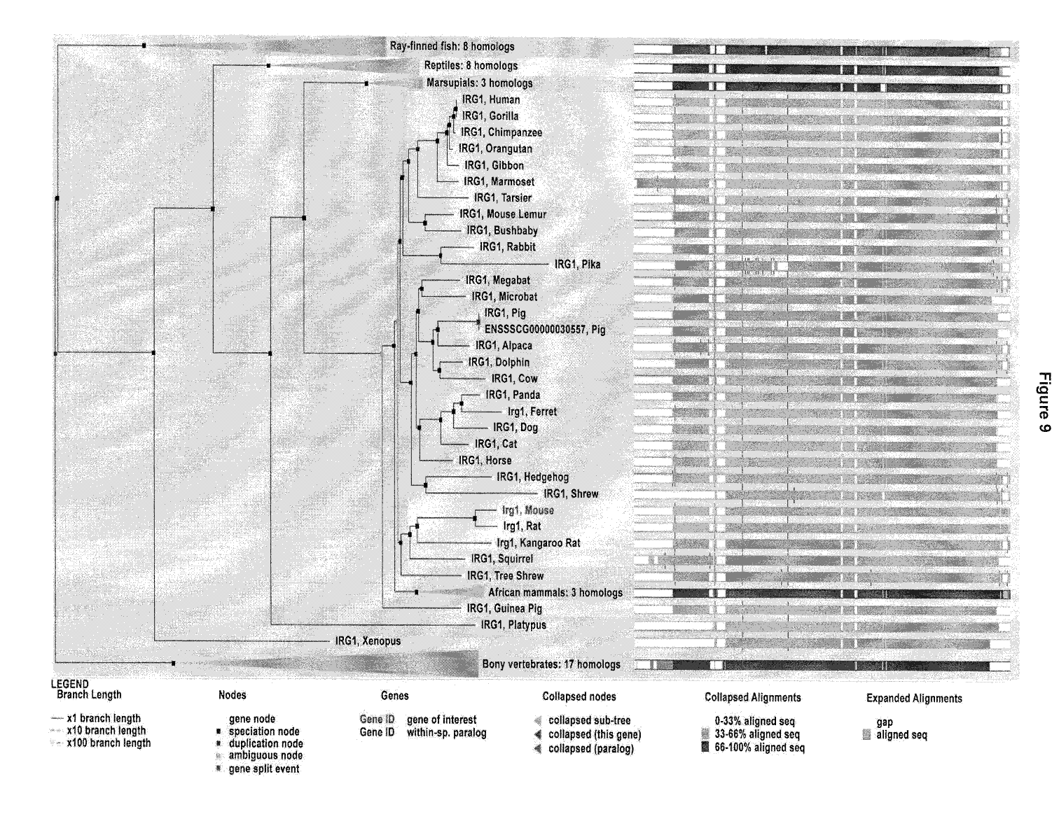

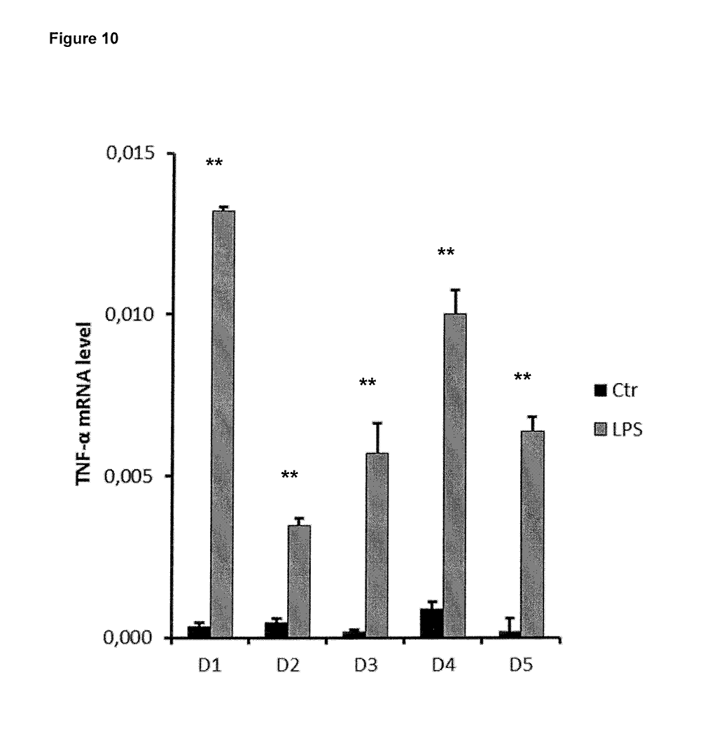

Since an Irg1 homologous gene is annotated in the human genome on chromosome 13, the inventors were interested to further analyze Irg1 expression and itaconic acid amounts in human immune cells. To investigate this, the inventors isolated CD14+ primary human monocytes from the blood of different donors, cultured them for differentiation into macrophages for 11 days and stimulated an inflammatory response with LPS (10 μg/ml) for 6 h. In line with their previous observations in mouse macrophages, the inventors observed that Irg1 expression in human peripheral blood mononuclear cells (PBMCs)-derived macrophages was highly up-regulated after LPS activation compared to resting conditions where Irg1 mRNA levels were almost undetectable (FIG. 4A). Our results are in accordance with those of Roach and colleagues (23), who analyzed LPS-activated PBMCs transcriptional profiles and observed Irg1 up-regulation compared to control conditions. At the metabolite level, itaconic acid amounts were highly increased under LPS-induced inflammatory conditions compared to resting cells in which the metabolite was measured in low amounts or below the detection limits (FIG. 4B). In line with the induction of itaconic acid production, the inventors observed elevated Irg1 expression (FIGS. 4C and 4D). A similar trend was also mirrored by the expression of TNF-α mRNA indicating that macrophages were activated (FIG. 10). Compared to the intracellular itaconic acid concentration in mouse immune cells, the concentration in human macrophages was two orders of magnitudes loer (8 mM vs. ˜60 μM).

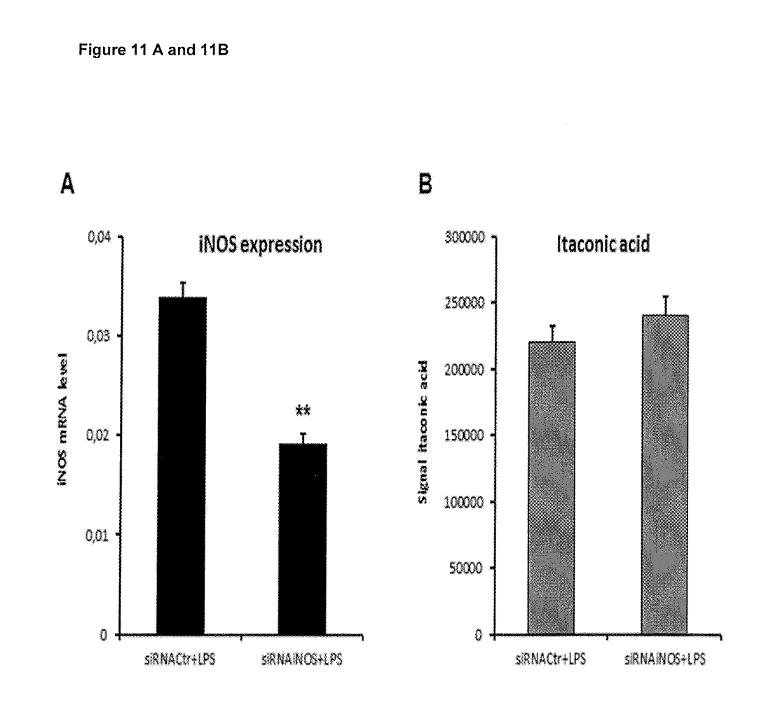

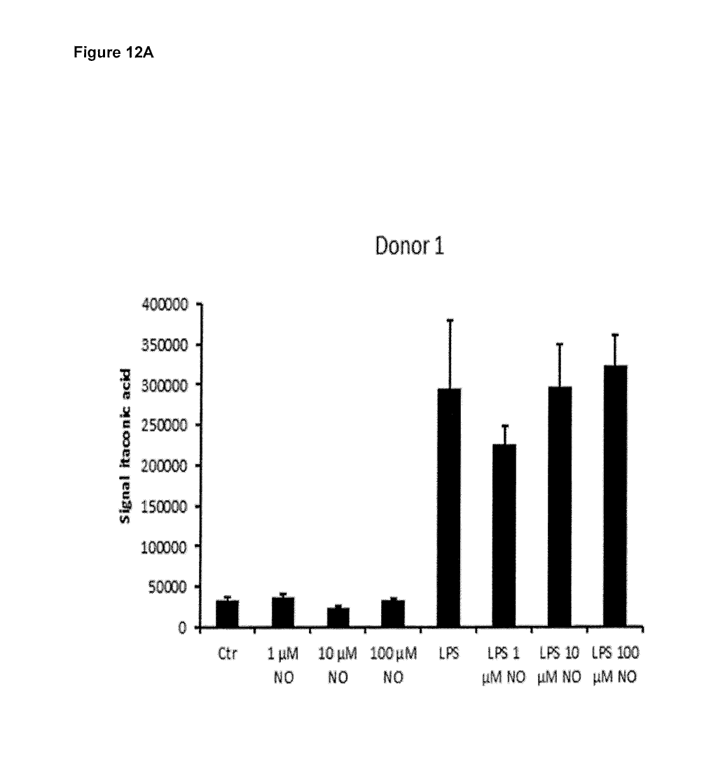

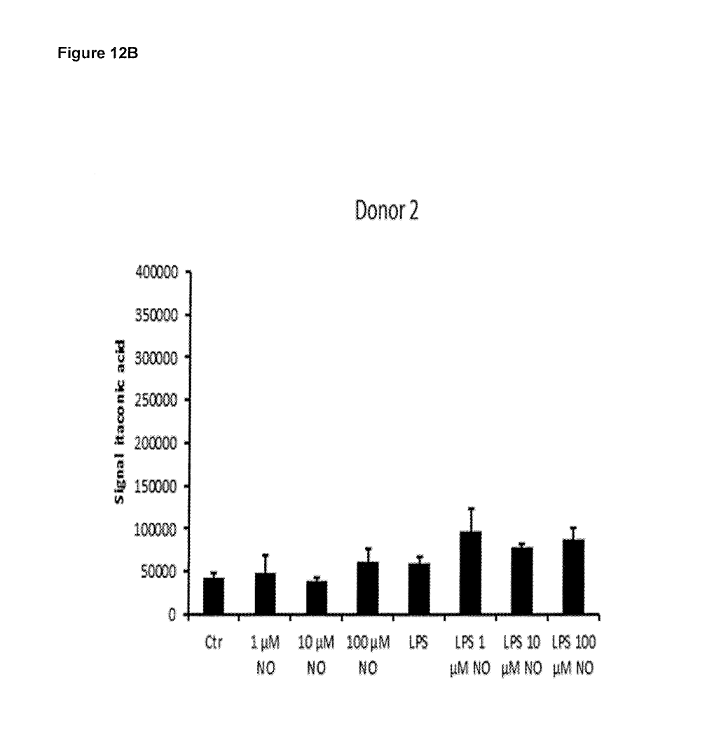

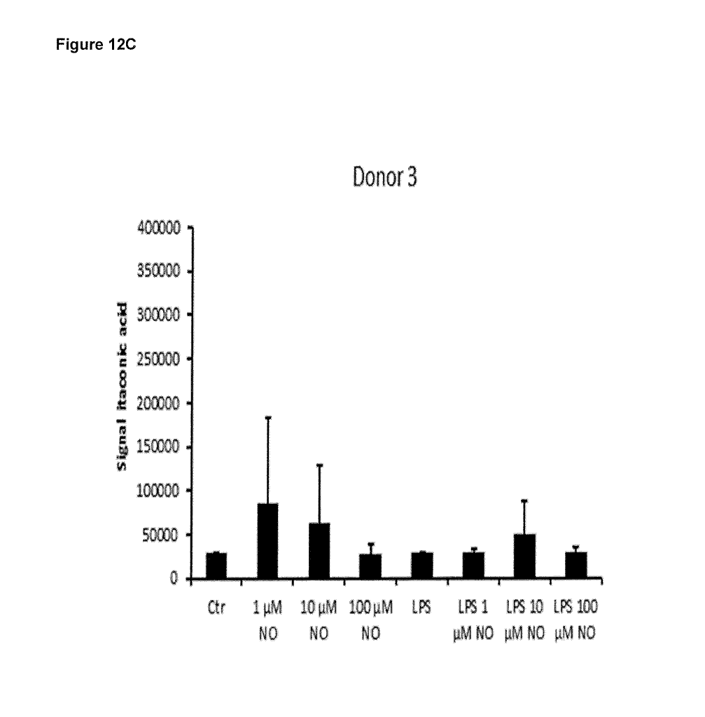

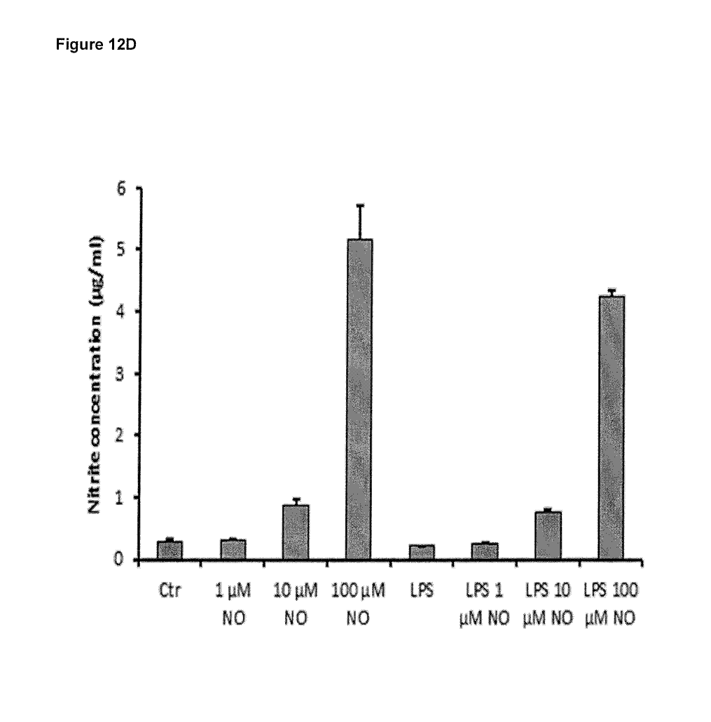

A major difference between mouse and human is the elevated production of nitric oxide in mouse macrophages under inflammatory conditions (24). It is well known that NO inhibits aconitase, the enzyme producing the itaconic acid precursor, cis-aconitate (25-27). To test whether the NO-mediated inhibition of aconitase has an effect on itaconic acid production, the inventors silenced the inducible nitric oxide synthetase (iNOS) gene to decrease NO levels in mouse macrophage (FIGS. 11A and 11B). On the other hand, the inventors treated human PBMCs-derived macrophages with the intracellular NO donor, diethylamine NONOate (28) to elevate the NO level in these cells (FIGS. 12A-C). In both cases, the inventors could not detect any effect on intracellular itaconic acid levels. Based on these results, the inventors assume that aconitase is not a rate-limiting step for itaconic acid synthesis.

Finally, the inventors transfected A549 human lung cancer cells with a pCMV6 plasmid expressing human Irg1 cDNA (phIrg1) to show CAD activity of human Irg1. The inventors observed high amounts of both Irg1 gene transcript and itaconic acid (0.044±0.0018 mM) 24 h post transfection (FIGS. 4E and 4F).

### Example 5

### In Vivo Irg1 Expression and Itaconic Acid Production

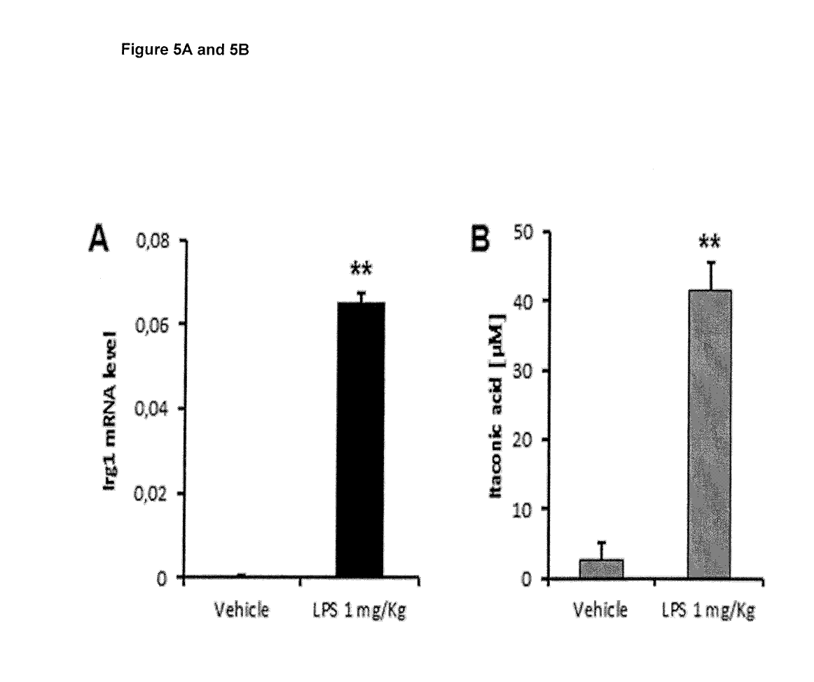

To confirm Irg1 expression and itaconic acid production in vivo, intraperitoneally injected C57B1/6 mice with LPS (1 mg/Kg) and harvested the peritoneal macrophages after 24 h. The inventors were able to measure high Irg1 mRNA expression levels correlating with high amounts of intracellular itaconic acid compared to the saline-injected mice (FIGS. 5A and 5B).

### Example 6

### Itaconic Acid Inhibits Bacterial Growth and Contributes to the Antimicrobial Activity of Mouse Macrophages

It has previously been shown that itaconic acid has an antimicrobial activity by inhibiting isocitrate lyase (ICL) (29, 30), an enzyme of the glyoxylate shunt. The glyoxylate shunt is not present in animals, but is essential for the survival of bacteria growing on fatty acids or acetate as the limiting carbon source (31). The strategy for survival during chronic stages of infection entails a metabolic shift in the bacteria's carbon source to C2 substrates generated by β-oxidation of fatty acids (31). Under these conditions, glycolysis is decreased and the glyoxylate shunt is significantly up-regulated to allow anaplerotic maintenance of the TCA cycle and assimilation of carbon via gluconeogenesis (32). Highly elevated levels of ICL are observed in Mycobacterium tuberculosis grown on C2 sources (33) and shortly after uptake into human macrophages (34). Furthermore, it has been shown that persistence of Mycobacterium tuberculosis in macrophages and mice requires the glyoxylate shunt enzyme ICL (35). In fact, Mycobacterium tuberculosis cannot persist in macrophages when both isoforms of ICL are genetically knocked out (36). As the glyoxylate shunt is exclusively found in prokaryotes, lower eukaryotes and plants, it represents a unique target for drug development (37).

To confirm the antimicrobial effect of itaconic acid on bacterial replication (30), the inventors cultured the pathogens Mycobacterium tuberculosis and Salmonella enterica (both known to express ICL for biosynthesis through the glyoxylate shunt) in liquid minimal medium supplemented with acetate as the unique carbon source to force the bacterial metabolism to use the glyoxylate shunt.

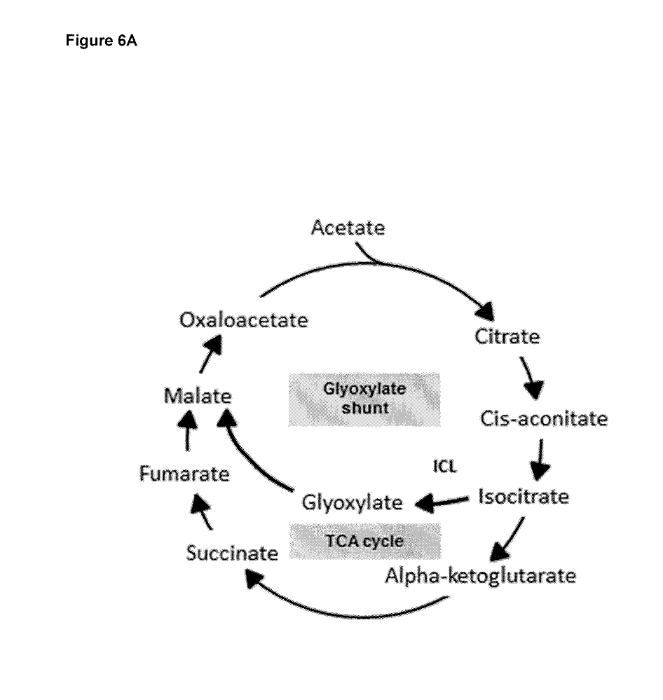

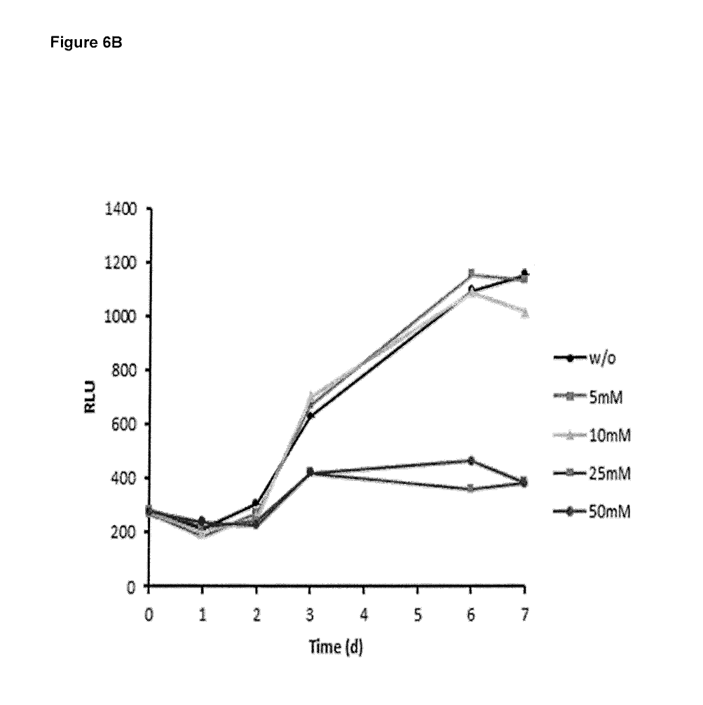

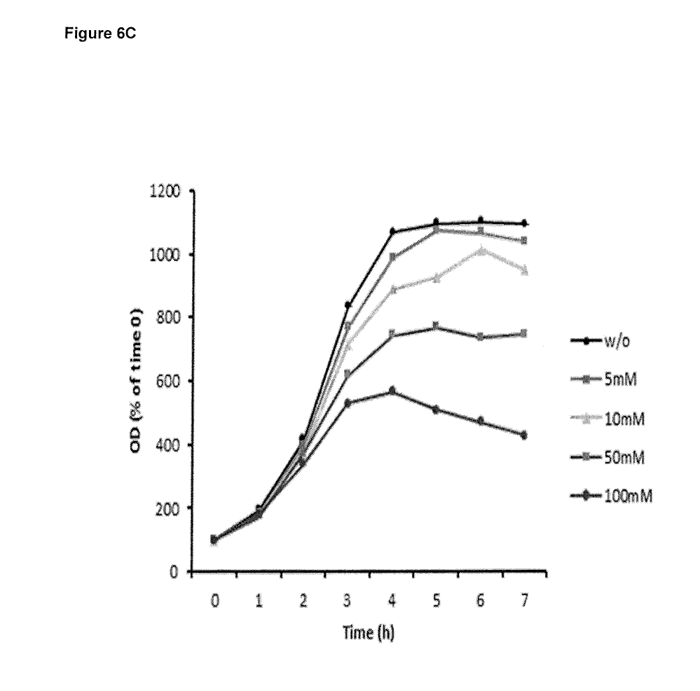

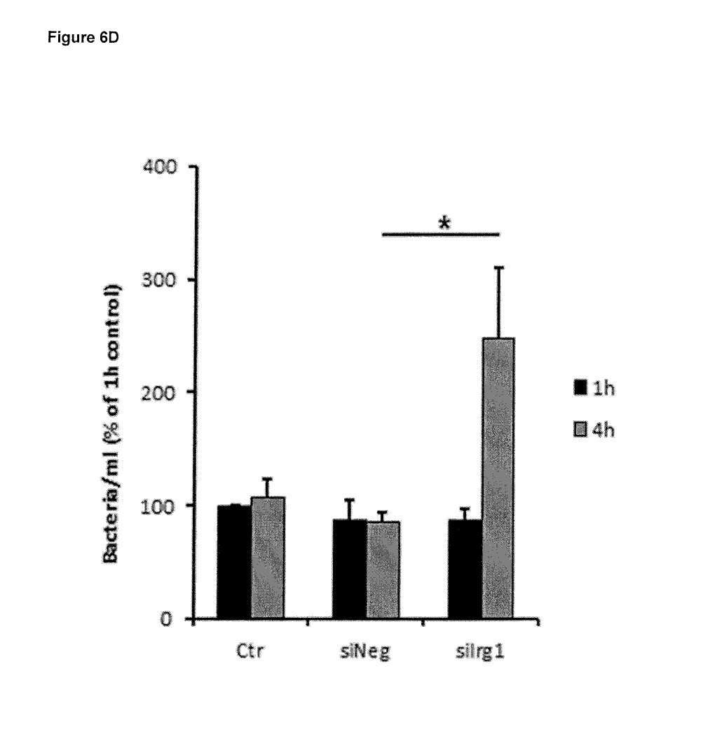

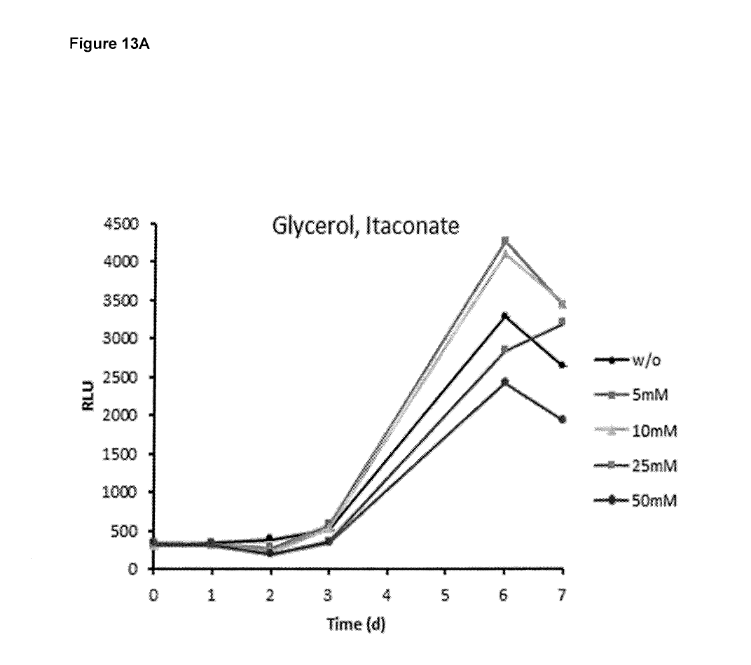

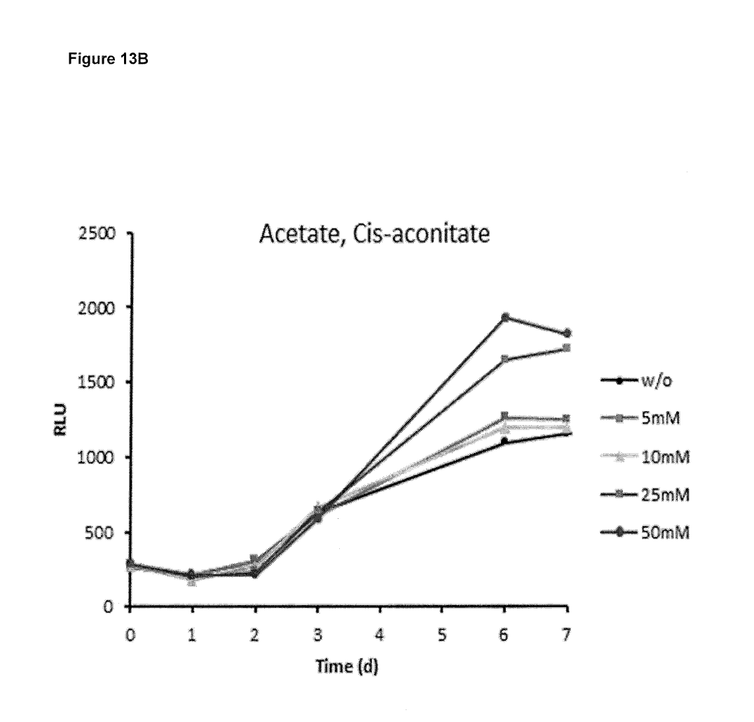

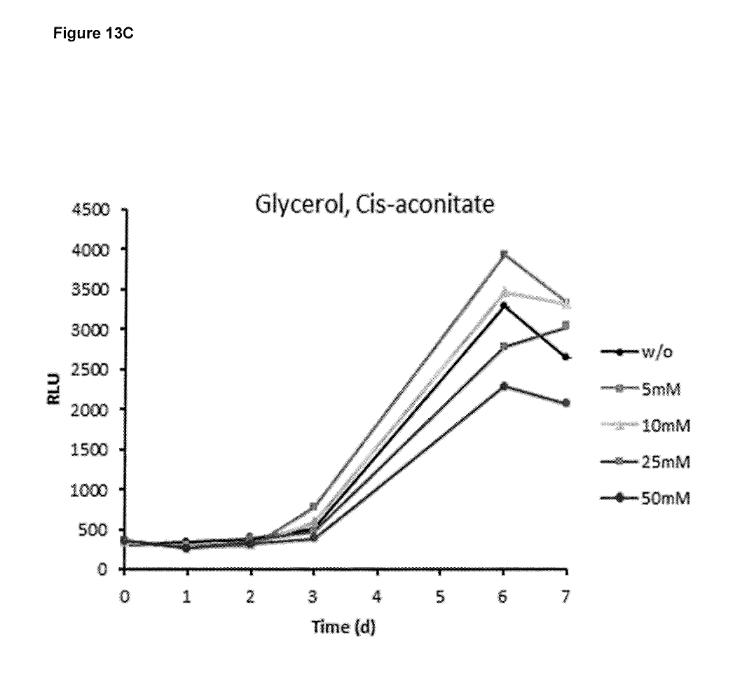

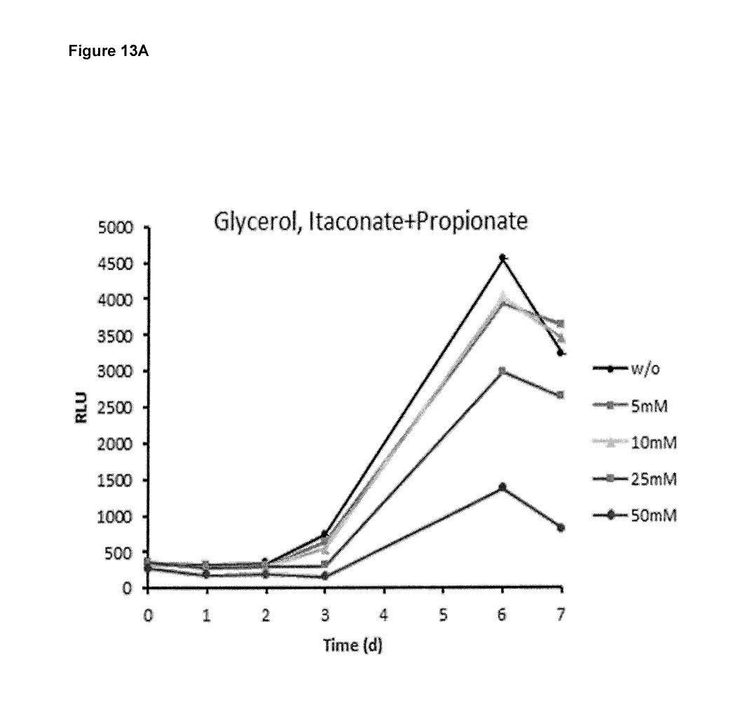

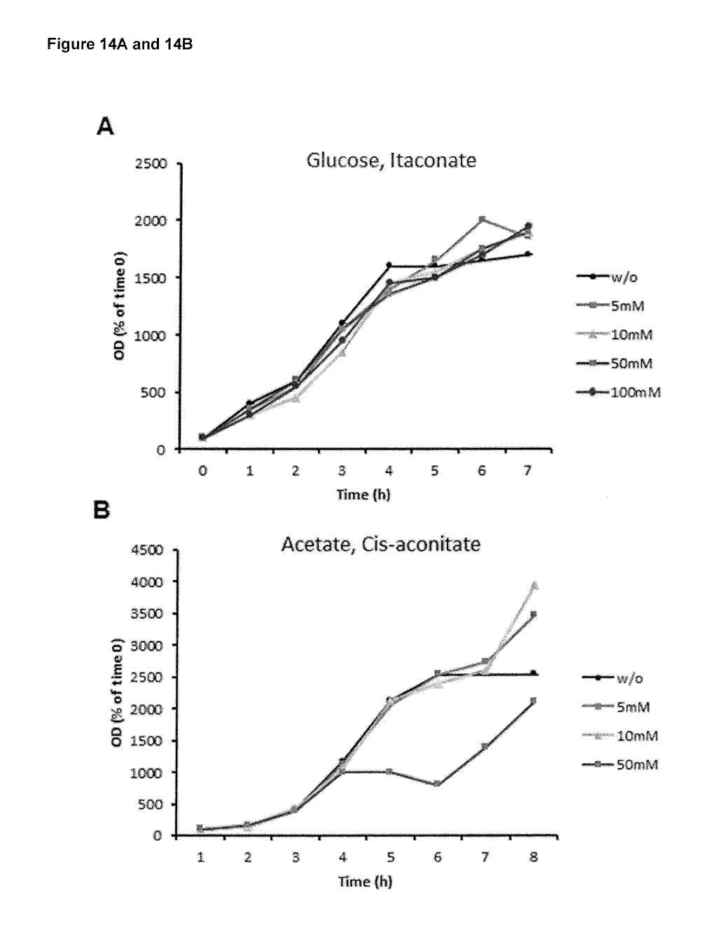

The inventors determined bacterial growth in this medium in the presence of increasing itaconic acid concentration and observed that the effective concentration of itaconic acid varies depending on the analyzed bacteria. The growth of M. tuberculosis in vitro was completely inhibited at 25-50 mM itaconic acid concentrations (FIG. 6B), while significant effects were already observed at 10 mM for S. enterica (FIG. 6C). To exclude secondary toxic effects of itaconic acid, the inventors measured the growth of the bacteria on glycerol or glucose as a carbon source. In this case bacterial metabolism does not rely on the glyoxylate shunt. Under these conditions, itaconic acid does not affect the bacterial growth (FIGS. 13A and 14A). To further demonstrate the specificity of the antimicrobial activity of itaconic acid, the inventors performed the same bacterial growth experiments, but by supplementing the medium with the itaconic acid precursor cis-aconitate. It was observed that bacteria grown in the presence of cis-aconitate elicited even a more pronounced growth at different concentrations of this metabolite in both bacteria (FIGS. 13B, 13C, 14B), thus indicating that these bacteria started to use cis-aconitate as carbon source, in strong opposition with the effect the inventors observed with itaconic acid.

It has been described that ICL exhibits an additional methylisocitrate lyase (MCL) activity in M. tuberculosis. MCL is required for the detoxification of propionyl-CoA through the methylcitrate cycle (38, 39). Propionyl-CoA accumulates during β-oxidation of odd-chain fatty acids and is produced from cholesterol of the host macrophages (40). As a result, inhibition of ICL in M. tuberculosis could have an additional toxic effect in the presence of propionate. To investigate the potential accentuated inhibition of the bacterial growth under these conditions, the inventors incubated M. tuberculosis in glycerol with 0.1 μM propionate and increasing concentrations of itaconic acid. Indeed, the combination of these two effects could inhibit M. tuberculosis growth already at 5-10 mM of itaconic acid (FIG. 13D), thus confirming that MCL activity of ICL is affected by the metabolite.

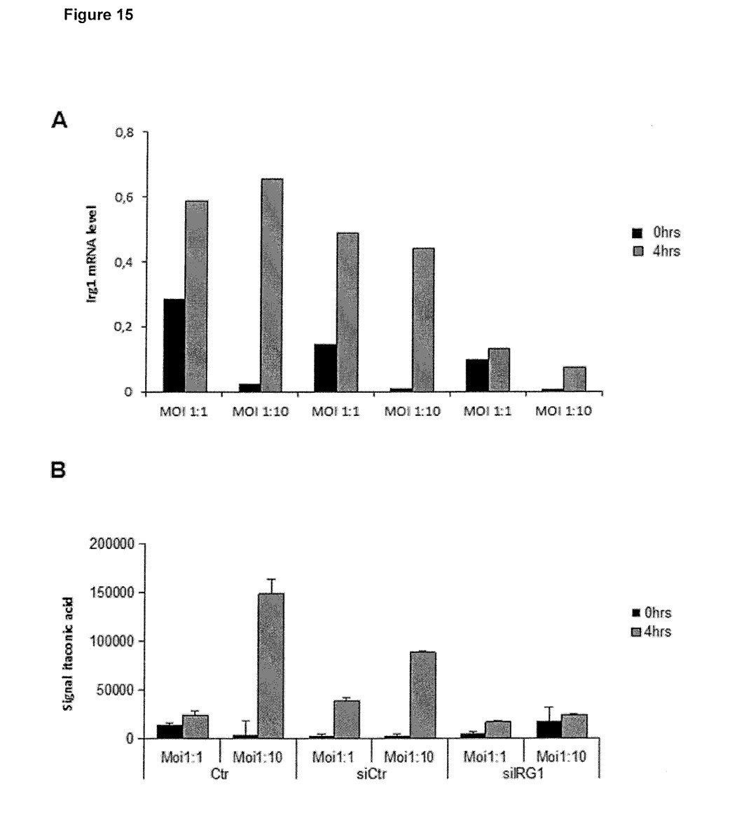

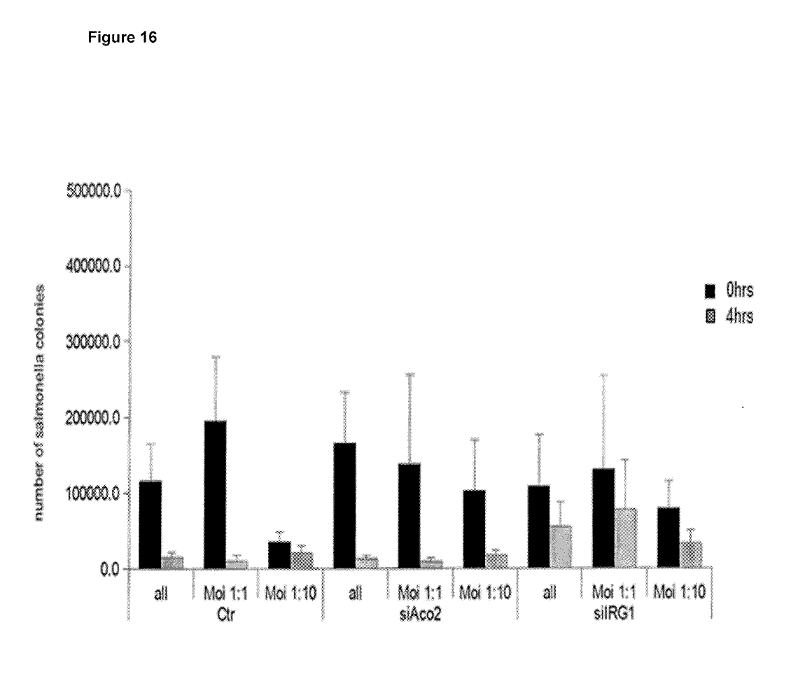

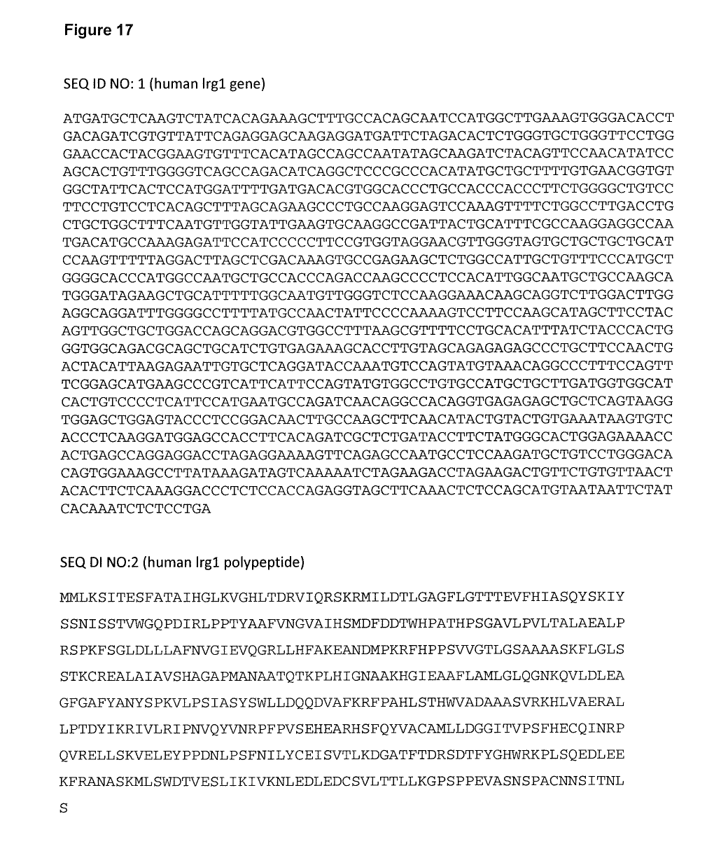

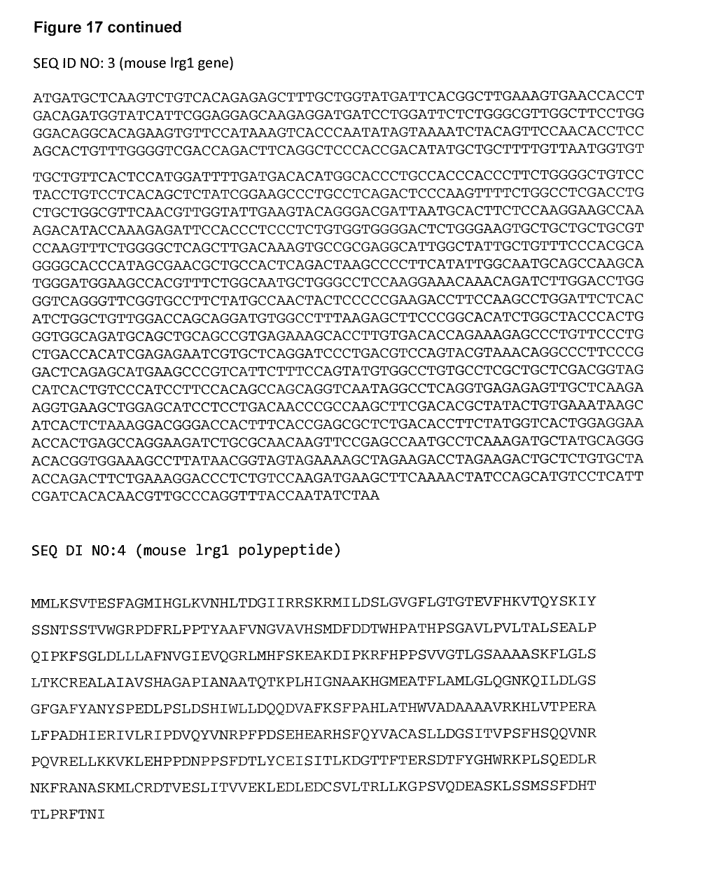

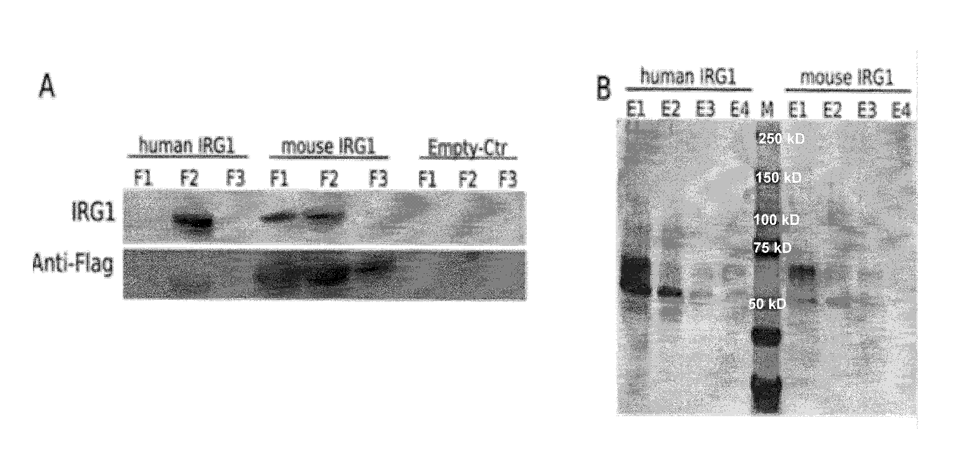

To further investigate the involvement of itaconic acid in the antimicrobial activity of macrophages, the inventors infected RAW264.7 cells with Salmonella enterica and consequently observed an increased Irg1 expression associated with high intracellular amounts of itaconic acid (FIGS. 15A and 15B). It was observed that silencing of Irg1 gene expression resulted in a decrease of intracellular itaconic acid concentration (FIG. 15B). The inventors detected a significantly larger number of intracellularly viable bacteria in macrophages treated with siRNA targeting Irg1 compared to those treated with an unspecific control siRNA or with siRNA targeting Aco2 4 h after infection (FIGS. 5D and 16).

Taken together, the results of the inventors demonstrated the importance indicate a role of Irg1 expression in macrophages during bacterial infection, thus contributing to their antimicrobial armature.

### Materials and Methods for Examples 1-6

### Cell Culture

Primary human monocytic CD14+ cells were isolated in two steps from blood samples provided by Red Cross Luxembourg. First, peripheral blood mononuclear cells (PBMCs) were separated in 50 ml Leucoseptubes (Greiner) through Ficoll-Paque™ Premium (GE Healthcare) density-gradient centrifugation at 1000 g for 10 minutes at room temperature with no brake. Second, CD14+ cells were purified with magnetic labeling. Therefore, 2 μl of CD14 Microbeads (Miltenyi Biotech) per 10⁷ PBMCs were incubated for 30 min at 4° C. followed by a positive LS column (Miltenyi Biotech) magnetic selection. The purified CD14+ cells were differentiated in six-well plates for 11 days in RPMI1640 medium without L-glutamine and phenol red (Lonza) supplemented with 10% human serum (A&E Scientific), 1% penicillin/streptomycin (Invitrogen) and 0.05% L-glutamine (Invitrogen). The medium was changed at day 4 and 7.

Four cell lines, specifically murine microglial BV-2 cells (42), murine macrophages RAW264.7 (43) (ATCC TIB-71), human epithelial A549 lung cancer cells (44) (ATCC CCL-185) and human HEK293T cells (45) were used.

### Cell Transfections.

The ON-TARGETplus SMARTpool containing four different siRNA sequences, all specific to murine Irg1 (siRNA Irg1), murine iNOS (siRNA iNOS), murine aconitase2 (siRNA Aco2) and the corresponding non-targeting control (siRNA Ctr) were designed and synthesized by Thermo Scientific Dharmacon.

RAW264.7 macrophages were transfected with Amaxa 4D-Nucleofector Device (Lonza), using the Amaxa SG cell line 4D Nucleofector Kit for THP-1 cells according to the manufacturer's instructions.

Briefly, transfection with siRNA complexes was carried out from pelleted and resuspended cells (1×10⁶ cells per condition). Transfection reagent and siRNA were prepared according to manufacturer's instructions (Amaxa). siRNAs were added at a final concentration of 100 nM. After the nucleofection processing using “RAW264.7 (ATCC) program” on the Nucleofection Device, the cells were seeded at a density of 1×10⁶ cells per well in 12-well plates in DMEM supplemented with 10% FBS and incubated during 24 h.

pCMV6-Irg1 overexpressing plasmid (4 μg, Mus musculus immune responsive gene 1 transfection-ready DNA, OriGene), in parallel with the empty plasmid (4 μg), was transfected into 1.5×10⁶ A549 cells using Lipofectamine 2000 (Invitrogen) and further incubated for 24 h. pCMV6-Entry-Irg1 plasmid was transfected into HEK293T cells by the jetPEI procedure as described previously (46) and further incubated for 48 h before extraction.

### Mouse Intraperitoneal Injection of LPS and Peritoneal Macrophages Isolation.

Three-4-month-old SJL mice were injected i.p. with LPS (1 mg/Kg) or with saline vehicle and were deeply anesthetized after 24 h by intraperitoneal injection of 50 mg/kg of Ketamine-HCl and 5 mg/kg Xylazine-HCl. Mice were then euthanized by cervical dislocation. Eight saline- and seven LPS-injected mice were used for peritoneal macrophages isolation. A small incision was made in the upper abdomen, and peritoneal macrophages were washed out with 4-5 ml ice-cold sterile PBS/mouse, and pooled into falcon tubes. The cell suspension was pelleted in a cooled centrifuge for 5 min at 250×g and the resulting pellet was worked up for metabolites and RNA extractions.

### Protein Purification and CAD Activity Assay.

HEK293T cells were extracted 48 h after transfection by scraping them into a lysis buffer containing 25 mM Hepes, pH 7.1 and 1× protease inhibitor cocktail (Roche). After two freeze/thaw cycles, cell extracts were incubated for 30 min on ice in the presence of DNAse I (200 U/ml extract; Roche Applied Science) and 10 mM MgSO₄. The crude cell extracts were centrifuged for 5 min at 16000×g (4° C.) and pellets were resuspended in lysis buffer for SDS-PAGE analysis. Flag-Irg1 was purified from the supernatant using the Flag®M purification kit, according to the manufacturer's instructions (Sigma Aldrich). About 3 mg protein were loaded onto 250 μl anti-Flag affinity resin and retained proteins were eluted with a solution containing 200 μg/ml Flag peptide (3×400 μl fractions). Protein purity was checked by SDS-PAGE analysis. Protein concentration was measured by the Bradford assay using Bradford reagent (Bio-Rad).

Cis-aconitate decarboxylase activity was measured by incubating cell extracts or purified protein fractions (10 μl) at 30° C. and for 40 min in a reaction mixture containing 25 mM Hepes, pH 7.1 and 1 mM cis-aconitate in a total volume of 100 μl. Reactions were stopped by addition of 900 μl methanol/water (8:1) mix. After 10 min centrifugation at 13200 rpm and 4° C., 100 μl of the supernatant were collected and evaporated under vacuum at −4° C. using a refrigerated CentriVapConcentrator (Labconco).

### RNA Isolation and Reverse-Transcription PCR (RT-PCR).

Total RNA was purified from cultured cells using the Qiagen RNeasy Mini Kit (Qiagen) as per manufacturer's instructions. First strand cDNA was synthesized from 0.5-2 μg of total RNA using Superscript III (Invitrogen) with 1 μl (50 μM)/reaction oligo(dT)₂₀ as primer. Individual 20 μl SYBR Green real-time PCR reactions consisted of 2 μl of diluted cDNA, 10 μl of 2×iQ™ SYBR Green Supermix (Bio-Rad), and 0.5 μl of each 10 μM optimized forward and reverse primers in 7 μL RNase-free water. Primer sequences designed using Beacon Designer software (Bio-Rad), provided by Eurogentec, or directly designed by Thermo Scientific, are available under request. For the human Irg1 primers, the NCBI/Primer-BLAST tool available at http://www.ncbi.nlm.nih.gov/tools/primer-blast/ was used. The PCR was carried out on a Light Cycler 480 (Roche Diagnostics), using a 3-stage program provided by the manufacturer: 10 min at 95° C. and 40 cycles of 30 sec at 95° C., 30 sec at 60° C., 30 sec at 72° C. followed by 10 sec 70-95° C. melting curves. All experiments included three no-template controls and were performed on three biological replicates with three technical replicates for each sample. For standardization of quantification, L27 was amplified simultaneously.

### SDS-PAGE and Western-Blotting Analysis.

Heat-denatured protein samples were separated on 10% SDS-polyacrylamide gels electrophoresis followed by transfer to nitrocellulose membranes 0.2 μm (Sigma). After blocking with 5% (w/v) dry milk in PBS, the membrane was incubated overnight at 4° C. in primary anti-Irg1 antibody from rabbit (Sigma) diluted 1:500 in 1% BSA/PBS with constant shaking. After three washing steps with PBS containing 0.1% Tween-20, the membrane was incubated with anti-rabbit antibody coupled to horseradish peroxidase and revealed by chemiluminescence using the Amersham ECL detection reagents (GE Healthcare).

### Gas Chromatography/Mass Spectrometry (GC/MS) Sample Preparation and Procedure.

Cells grown on 6-well plates were washed with 1 ml saline solution and quenched with 0.4 ml −20° C. methanol. After adding an equal volume of 4° C. cold water cells were collected with a cell scraper and transferred in tubes containing 0.4 ml −20° C. chloroform. The extracts were vortexed at 1400 rpm for 20 min at 4° C. and centrifuged at 16000 g for 5 min at 4° C. 0.3 ml of the upper aqueous phase was collected in specific GC glass vials and evaporated under vacuum at −4° C. using a refrigerated CentriVap Concentrator (Labconco). The metabolite extractions of cells grown on 12-well plates were performed using half of the volumes.

The interphase was centrifuged with 1 ml −20° C. methanol at 16000 g for 5 min at 4° C. The pellet was used for RNA isolation.

Metabolite derivatization was performed using an Agilent Autosampler. Dried polar metabolites were dissolved in 15 μl of 2% methoxyamine hydrochloride in pyridine at 45° C. After 30 minutes an equal volume of MSTFA (2,2,2-trifluoro-N-methyl-N-trimethylsilyl-acetamide)+1% TMCS (chloro-trimethyl-silane) were added and hold for 30 min at 45° C. Metabolites extracted out of 12-well plates were derivatized using half of the reagent volumes. GC/MS analysis is described in Supplemental Information section.

### Glucose Labeling Assay.

RAW264.7 macrophages were seeded at a density of 1×10⁶ per well in 12-well plates in DMEM medium supplemented with 10% FBS and 1% penicillin/streptomycin at 37° C. with 5% CO₂. After 24 h, the medium was changed to DMEM containing uniformly labeled 25 mM [U-¹³C] glucose (Cambridge Isotope). Simultaneously, the cells were activated with 10 ng/ml LPS. After 6 h of incubation, the metabolites were extracted.

Salmonella enterica Growth Analysis.

Salmonella enterica serovar Typhimurium bacteria were grown in liquid medium as detailed in the text in the presence different concentrations of itaconic acid or cis-aconitate (5, 10, 50, 100 mM). Growth was measured as optical density (OD) at indicated time points.

Mycobacterium tuberculosis Growth Analysis.

GFP-expressing Mycobacterium tuberculosis H37Rv bacteria (47) were generated using the plasmid 32362:pMN437 (Addgene), kindly provided by M. Niederweis (University of Alabama, Birmingham, Ala.) (48). 1×10⁶ bacteria were cultured in 7H9 medium supplemented with different carbon sources as indicated in a total volume of 100 μl in a black 96 well plate with clear bottom (Corning Inc, Corning, N.Y.) sealed with an air-permeable membrane (Porvair Sciences, Dunn Labortechnik, Asbach, Germany). Growth was measured as relative light units (RLU) at 528 nm after excitation at 485 nm in Fluorescence microplate reader (Synergy 2, Biotek, Winooski, Vt.) at indicated time points.

### Macrophages Bacterial Phagocytosis and Killing Assay.

Untransfected or transfected RAW264.7 macrophages (with unspecific siRNA, IRG1 specific siRNA or mitochondrial Aconitase specific siRNA) were seeded at a density of 25×10⁴ per well in 48-well plates in 250 μl DMEM medium complemented with 10% FBS at 37° C. with 5% CO₂. After 24 hours, the cells were infected with Salmonella enterica serovar Thyphimurium at a multiplicity of infection (MOI) of 1:10 (one bacteria per ten macrophages) or 1:1 (one bacteria per one macrophage) and incubated for 1 h at 37° C. with 5% CO₂. Macrophages were then washed with sterile PBS and re-suspend in DMEM medium complemented with 10% FBS and 100 ug/ml gentamicin to kill non ingested bacteria and further incubated for 1 h (this was considered as timepoint 0 h) or 4 h (timepoint 4 h) at 37° C. with 5% CO₂. After washing with sterile PBS, macrophages were disrupted for 15 min with 250 μl dH₂O to release intracellular bacteria. The amount of viable intracellular bacteria was determined by plating on LB-(Luria-Bertani)-Agar plates using four dilutions from 1:10 up to 1:10000 and incubation O/N at 37° C. For metabolite extraction, macrophages were seeded at a density of 75×10⁴ per well in 12-well plates. Intracellular metabolites were extracted and mRNA isolated at timepoint 0 h and timepoint 4 h for GC/MS measurements and RT-PCR, respectively. All conditions were performed in technical triplicates.

### Statistical Analysis.

For comparison of means between two different treatments the statistical analysis was done by the Student's t-test unless otherwise indicated. Error bars indicate SD or SEM as specified in the text.

### Mice.

All animal procedures have been performed according to the European Guidelines for the use of animals in research (86/609/CEE). All efforts were made to minimize suffering. All animals have been raised and crossed in an indoor animal house in a 12 h light/dark cycle and have been provided with water and food ad libitum.

### Cell Culture.

Mixed glial cell cultures were prepared from the brains of new born C57BL/6 mice. After carefully removing meninges and large blood vessels, the brains were pooled and then minced in cold phosphate buffered saline (PBS) solution. The tissue was mechanically dissociated with Pasteur pipettes and the resultant cell suspension was passed through a 21G hypodermic needle. After washes and centrifugations, the mixed glial cells were plated into poly-D-lysine (PDL, Sigma) coated 6-well plates (2 brains per 6-well plate) in Dulbecco's modified Eagle's medium (DMEM) (Invitrogen) supplemented with 100 U/ml penicillin, 100 mg/ml streptomycin (Sigma) and 10% heat-inactivated foetal bovine serum (FBS, Invitrogen) in a water-saturated atmosphere containing 5% CO2 at 37° C. The medium was replaced every 3-4 days. After 7-10 days, when the cultures reached confluence, microglia were detached by a 30 min shaking on a rotary shaker (180 rpm). Detached cells, mainly microglia (>95%), were then plated in multi-well plates in conditioned medium and further incubated for 3 days.

BV-2, HEK293T and RAW264.7 cell lines were maintained in DMEM with or without sodium pyruvate, supplemented with 10% heat-inactivated FBS (South American, Invitrogen). No antibiotics were used for BV-2, 1% penicillin/streptomycin were used for RAW264.7 and HEK293T cells.

A549 cells were cultivated in DMEM without sodium pyruvate, supplemented with 10% heat-inactivated FBS and 1% penicillin/streptomycin. Cells were grown and maintained according to standard cell culture protocols and kept at 37° C. with 5% CO₂.

For experiments, BV-2, RAW264.7 and A549 cells were seeded into multi-well plates at a density of 0.5×10⁵ (BV-2) and 1.0×10⁵ (RAW264.7 and A549) cells/well (six-well plates). After 3 days of culture, the cells were activated adding specific stimuli to the culture medium.

Lipopolysaccharide (LPS 055:B5 from Escherichia coli, Sigma) was added at specified time points and at different doses in mouse primary microglia (1 ng/ml), BV-2 and RAW264.7 (10 ng/ml) or PBMCs-derived macrophages (10 μg/ml) to obtain similar activation states because of the differences in sensitivity between murine primary cultures and cell lines as well as between mouse and human cells.

### GC/MS Analysis.

GC/MS analysis was performed using an Agilent 6890 GC equipped with a 30 m DB-35MS capillary column. The GC was connected to an Agilent 5975C MS operating under electron impact (EI) ionization at 70 eV. The MS source was held at 230° C. and the quadrupole at 150° C. The detector was operated in scan mode and 1 μl of derivatized sample was injected in splitless mode. Helium was used as carrier gas at a flow rate of 1 ml/min. The GC oven temperature was held on 80° C. for 6 min and increased to 300° C. at 6° C./min. After 10 minutes the temperature was increased to 325° C. at 10° C./min for 4 min. The run time of one sample was 59 min.

### NO Donor Treatments.

Human PBMCs were seeded and differentiated into macrophages as described above. Diethylamine NONOate (DEA NONOate, Sigma), an intracellular NO donor, was added at different concentrations (1, 10, 100 μM) alone or together with LPS (100 μg/ml). After 12 h of incubation, the metabolites were extracted.

### Griess Nitrite Assay.

After 12 h, 180 μl of medium was harvested and combined with 20 μl of 1 mM NaOH on ice to stop the dissociation reaction. Levels of nitrite formed from the reaction with H₂O were determined using the Griess assay. In brief, 50 μl of medium sample or nitrite IC standard (Sigma) was pipetted in triplicate in a 96-well plate. To each well, equal volumes of 1× Griess Reagent (Sigma) were added. Absorbance was read at 540 nm and nitrite concentrations were calculated.

### Sequence Alignment.

Multiple sequence alignment of Cis-aconitic acid decarboxylase (Aspergillus terreus), Immune-responsive gene 1 protein homolog (human), Immune-responsive gene 1 protein (mouse) and Imunodisuccinate Epimerase (Agrobacterium tumefaciens) was performed using MAFFT version 6 (50, 51) and visualized with ESPript (52). Sequences were obtained from UniProt Knowledgebase (UniProtKB) with the following accession numbers: B3IUN8 (CAD1), A6NK06 (IRG1 human) P54987 (Irg1 mouse) and Q1L4E3 (IDS epimerase).

### Example 7

### Enzymatic Characterization of IRG1 and CAD

Industrially, itaconic acid is produced using the fungus Aspergillus terreus. Since high intracellular itaconic acid levels in mammalian cells were found, it is possible that mammalian IRG1 is able to produce itaconic acid at higher rates than CAD.

Since the enzymatic function of IRG1 was described in the present invention for the first time, kinetic parameters of the IRG1 protein characterization are not known. However, the CAD enzyme, which catalyzes itaconic acid production in Aspergillus terreus and is currently used for industrial itaconic acid production (Steiger et al., 2013), was first purified and characterized by Dwiarti (62). The authors determined the Michaelis-Menten constant (K_{M}) for the substrate cis-aconitic acid at pH=6.2 and 37° C. with K_{M}=2.45 mmol·*I⁻¹ (62). To compare the kinetic properties of CAD and IRG1, enzyme activity assays were performed with purified murine and human IRG1 protein.

### IRG1 Protein Purification

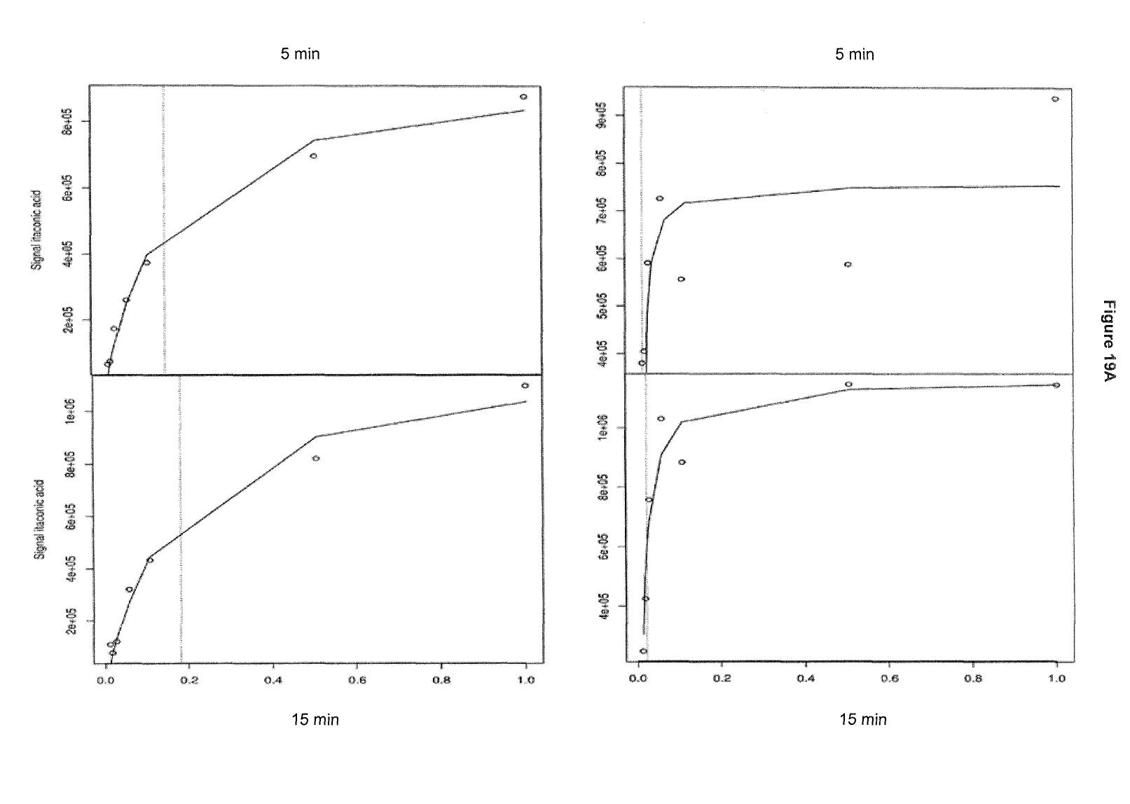

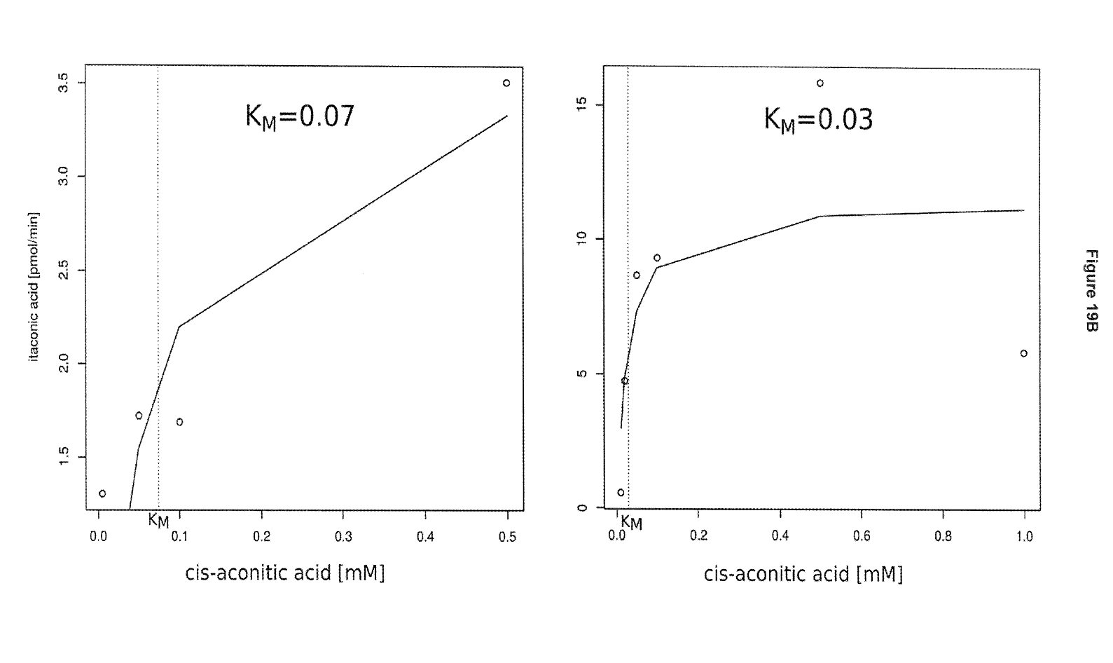

To produce high amounts of Flag-tagged IRG1 protein needed for the enzyme activity assays, HEK 293T were transfected cells with pCMV6-Entry expression plasmid containing either a human IRG1 or a murine Irg1 coding sequence. An empty plasmid was used as negative control and purified the proteins by loading them onto an affinity resin. To confirm the presence of purified human and murine IRG1 protein, a silver staining and a Western Blot analysis using specific IRG1 and Flag antibodies we performed (see FIG. 18)

Analysis by Western Blot using Anti-Flag and specific IRG1 antibodies showed purified protein with a molecular mass of ˜55 kDa for human IRG1 in elution fractions F1 and F2, while no signal was detected in protein extracts from control empty plasmids (see FIG. 18A). Moreover, mouse and human IRG1 proteins were determined as single band with the same molecular mass using silver staining in SDS gel (see FIG. 18B). The size of the protein bands was in line with the published size of human and murine IRG1 proteins of 53 kDa (UniProtKB). Therefore, the presence of purified human and murine IRG1 proteins could be confirmed.

### Kinetic Parameters of IRG1

The purified proteins were then used for enzyme activity assays. Cis-aconitic acid was used as a substrate at concentrations in the range of 0 to 1 mmol*I⁻¹ at pH=6.2 and 37° C. To determine the time dependent itaconic acid production, itaconic acid level was measured after 5 and 15 min of incubation. To determine the correlation between itaconic acid production and IRG1 activity, the substrate concentrations were plotted against the rate of itaconic acid formation.

An increasing itaconic acid production was observed over time in the presence of either murine or human IRG1 and cis-aconitic acid as substrate (see FIGS. 19A and 19B). These data confirmed the enzymatic function of murine and human IRG1 for itaconic acid production. Increasing itaconic acid signals correlated with increasing substrate concentrations until reaching the maximal reaction velocity. To compare the kinetics of IRG1 and CAD, the kinetic parameter K_{M} was calculated.

Michaelis-Menten constant (K_{M}) of CAD, murin and human IRG1 for itaconic acid formation using cis-aconitic acid substrate

| Protein | Organism | K_{M}[mmol · l⁻¹] |
|---|---|---|
| IRG1^{a} | mouse | 0.07 |
| IRG1^{a} | human | 0.03 |
| CAD^{b} | Aspergillus terreus | 2.45^{c} |

^{a}= IRG1,
^{b}= CAD,
^{c}= see Ref. 62

A K_{M} of 0.07 mmol*I⁻¹ for the murine protein and a K_{M} of 0.03 mmol*I⁻¹ for the human protein were determined. Compared to the fungal CAD with a K_{M} of 2.45 mmol*I⁻¹ (62), K_{M} of mammalian IRG1 was two orders of magnitudes lower. A lower K_{M} means a higher binding affinity of the enzyme to the substrate cis-aconitic acid. Thus, IRG1 has a higher substrate affinity indicated by a lower mammalian Kₘ. Therefore, the use of IRG1 instead of CAD amino acid sequence might significantly increase itaconic acid production.

### Material and Methods for Example 7

Proteins produced in HEK293FT cells, which have been transfected with human and mouse pCMV6-Irg1 overexpression plasmid as well as pCMV6-Entry plasmid, were purified, separated on SDS-Page, detected with western blotting or silver staining and characterized with in-vitro enzyme assays.

### Plasmids

For protein characterization, mouse and human pCMV6-IRG1 overexpression plasmids were cloned with common molecular biological techniques. For murine and human pCMV6-IRG1 overexpression plasmids, murine and human IRG1-sequence was cloned into pCMV6-Entry donation plasmid (OriGene). PCMV6-Entry plasmid contained a peptide sequence needed for expression of FLAG-tagged proteins for protein purification.

### Cell Transfection

HEK293FT cells were cultured in DMEM medium (D-6429, Sigma) supplemented with 10% FBS (v/v) and 1% P/S (v/v), 1% L-Glutamine (v/v) 200 mmol*I⁻¹, 1% non-essential amino acids (100×) (v/v) and 1% G418 (v/v) disulfate solution. Cell layers were dispersed with Trypsin for 2 min at 37° C.

For protein production, HEK293FT cells were transfected using Lipofectamine 2000 (Invitrogen). Cells were seeded at a density of 6×10⁶ cells on petri plates in growth medium without G418 disulfate solution and antibiotics and transfected with 3 μg expression plasmid. 48 h after transfection, lentiviruses were harvested and proteins extracted.

### Western Blotting

Separated proteins were transferred to 0.2 μm nitrocellulose membranes and incubated with antibodies against IRG1 and FLAG-tag. All incubation steps were carried out with constant shaking. Washing steps and blocking were performed in Tween supplemented 0.1% PBS.

The membrane was blocked with 5% dry milk (w/v) for 1 h at room temperature, washed three times and incubated overnight at 4° C. with the primary antibody against IRG1 (anti-IRG1 hpa 040143, Sigma) diluted 1:250 in PBS supplemented with 1% BSA (w/v). The membrane was then washed three times and incubated with the second antibody anti-rabbit coupled to horseradish peroxidase (HRP) (sc-2004, Santa Cruz Biotechnology) diluted 1:5000 in 5% dry milk in 0.1% PBS-Tween for 1 h at room temperature.

After stripping with Restore Western Blot Stripping Buffer (Thermo Scientific) for 10 min, washing 3-times and blocking with 3% dry milk (w/v) for 1 h, the membrane was incubated for 3 h at room temperature with the primary anti-FLAG antibody (Sigma) diluted 1:1000 in 3% dry milk (w/v) in 0.1% PBS-Tween. The membrane was then washed three times with 0.1% TBS-Tween and incubated with the second antibody anti-mouse coupled to horseradish peroxidase (HRP) (sc-2005, Santa Cruz Biotechnology) diluted 1:8000 in 3% dry milk (w/v) in 0.1% TBS-Tween for 1 h at room temperature.

Chemiluminescence of secondary antibodies was detected using Amersham ECL detection reagents (GE Healthcare) with Odyssey 2800 (Licor).

### Silver Staining

Separated proteins were detected by silver staining using Silver Quest staining kit (Invitrogen) according to manufacturer's instructions. Deviating from the protocol, only 20 ml of sensitizing, staining and developing solutions were used. Fixing solution contained 30% methanol (v/v) and 20% acetic acid (v/v) in water. All incubation steps were carried out at room temperature with constant shaking.

### CAD Activity Assay

Cis-aconitate decarboxylase (CAD) activity assay was performed at 37° C. with 300 μl reaction volume containing 10% purified enzyme solution (v/v), 25 mmol*I⁻¹ HEPES (pH=6.2) and 8 different substrate concentrations with pH=6.2: 0, 5, 10, 20, 50, 100, 200, 500 and 100 μmol*I⁻¹. Enzyme solutions were used from F1 as well as F2 and cis-aconitic acid and citric acid were used as substrates. Sampling took place 5 min and 15 min after enzyme supplementation. 95 μl enzyme solution were transferred into sampling tubes containing 230 μl methanol at −20° C. and centrifuged for 10 min at 4° C. with 16,000×g. 290 μl were dried in glass vials under vacuum and analyzed with GC-MS. Additionally, standard curves were prepared with cis-aconitic acid, citric acid and itaconic acid at concentrations with 10, 40, 80 and 100 μmol*I⁻¹. The kinetic parameter Kₘ of the purified enzymes was determined with Michaelis-Menten model using the statistical tool R (R Development Core Team, 2011).

### REFERENCES

1. David S, Kroner A (2011) Repertoire of microglial and macrophage responses after spinal cord injury. Nat Rev Neurosci 12:388-99.
2. Martinez F O, Sica A, Mantovani A, Locati M (2008) Macrophage activation and polarization. Front Biosci 13:453-461.
3. Mantovani A, et al. (2004) The chemokine system in diverse forms of macrophage activation and polarization. Trends Immunol 25:677-686.
4. Michelucci A, Heurtaux T, Grandbarbe L, Morga E, Heuschling P (2009) Characterization of the microglial phenotype under specific pro-inflammatory and anti-inflammatory conditions: Effects of oligomeric and fibrillar amyloid-beta. J Neuroimmunol 210:3-12.
5. Tangsudjai S, et al. (2010) Involvement of the MyD88-independent pathway in controlling the intracellular fate of Burkholderia pseudomallei infection in the mouse macrophage cell line RAW 264.7. Microbiol Immunol 54:282-290.
6. Degrandi D, Hoffmann R, Beuter-Gunia C, Pfeffer K (2009) The proinflammatory cytokine-induced IRG1 protein associates with mitochondria. J Interferon Cytokine Res 29:55-67.
7. Thomas D M, Francescutti-Verbeem D M, Kuhn D M (2006) Gene expression profile of activated microglia under conditions associated with dopamine neuronal damage. FASEB J 20:515-517.
8. Basler T, Jeckstadt S, Valentin-Weigand P, Goethe R (2006) Mycobacterium paratuberculosis, Mycobacterium smegmatis, and lipopolysaccharide induce different transcriptional and post-transcriptional regulation of the IRG1 gene in murine macrophages. J Leukoc Biol 79:628-638.
9. Gautam A, et al. (2011) Interleukin-10 alters effector functions of multiple genes induced by Borrelia burgdorferi in macrophages to regulate Lyme disease inflammation. Infect Immun 79:4876-4892.
10. Matulova M, et al. (2012) Characterization of chicken spleen transcriptome after infection with Salmonella enterica serovar Enteritidis. PLoS One 7:e48101.
11. Smith J, et al. (2011) Systems analysis of immune responses in Marek's disease virus-infected chickens identifies a gene involved in susceptibility and highlights a possible novel pathogenicity mechanism. J Virol 85:11146-11158.
12. Lee C G, Jenkins N A, Gilbert D J, Copeland N G, O'Brien W E (1995) Cloning and analysis of gene regulation of a novel LPS-inducible cDNA. Immunogenetics 41:263-270.
13. Cheon Y P, Xu X, Bagchi M K, Bagchi I C (2003) Immune-responsive gene 1 is a novel target of progesterone receptor and plays a critical role during implantation in the mouse. Endocrinology 144:5623-5630.
14. Chen B, Zhang D, Pollard J W (2003) Progesterone regulation of the mammalian ortholog of methylcitrate dehydratase (immune response gene 1) in the uterine epithelium during implantation through the protein kinase C pathway. Mol Endocrinol 17:2340-2354.
15. Mor G, Cardenas I, Abrahams V, Guller S (2011) Inflammation and pregnancy: the role of the immune system at the implantation site. Ann N Y Acad Sci 1221:80-87.
16. Li H, et al. (2006) Different neurotropic pathogens elicit neurotoxic CCR9- or neurosupportive CXCR3 expressing microglia. J Immunol 177:3644-3656.
17. Lohkamp B, Bäuerle B, Rieger P G, Schneider G. (2006) Three-dimensional structure of iminodisuccinate epimerase defines the fold of the MmgE/PrpD protein family. J Mol Biol 362:555-566.
18. Kanamasa S, Dwiarti L, Okabe M, Park E Y (2008) Cloning and functional characterization of the cis-aconitic acid decarboxylase (CAD) gene from Aspergillus terreus. Appl Microbiol Biotechnol 80:223-229.
19. Strelko C L, et al. (2011) Itaconic acid is a mammalian metabolite induced during macrophage activation. J Am Chem Soc 133:16386-16389.
20. Bentley R, Thiessen C P (1957) Biosynthesis of itaconic acid in Aspergillus terreus. I. Tracer studies with C14-labeled substrates. J Biol Chem 226:673-687.
21. Okabe M, Lies D, Kanamasa S, Park E Y (2009) Biotechnological production of itaconic acid and its biosynthesis in Aspergillus terreus. Appl Microbiol Biotechnol 84:597-606.
22. Bonnarme P, et al. (1995) Itaconate biosynthesis in Aspergillus terreus. J Bacteriol 177:3573-3578.
23. Roach J C, et al. (2007) Transcription factor expression in lipopolysaccharide-activated peripheral blood-derived mononuclear cells. Proc Natl Acad Sci USA 104:16245-16250.
24. Schroder K, et al. (2012) Conservation and divergence in Toll-like receptor 4-regulated gene expression in primary human versus mouse macrophages. Proc Natl Acad Sci USA 109:E944-E953.
25. Gardner P R, Costantino G, Szabó C, Salzman A L (1997) Nitric oxide sensitivity of the aconitases. J Biol Chem 272:25071-25076.
26. Drapier J C, Hibbs J B (1996) Aconitases: a class of metalloproteins highly sensitive to nitric oxide synthesis. Methods Enzymol 269:26-36.
27. Stadler J, et al. (1991) Effect of exogenous and endogenous nitric oxide on mitochondrial respiration of rat hepatocytes. Am J Physiol 260:C910-C916.
28. Kovaleva V, Berezhnaya E, Komandirov M, Rudkovskii M, Uzdensky A (2013) Involvement of nitric oxide in photodynamic injury of neurons and glial cells. Nitric Oxide 29C:46-52.
29. Patel T R, McFadden B A (1978) Caenorhabditis elegans and Ascaris suum: inhibition of isocitrate lyase by itaconate. Exp Parasitol 44:262-268.
30. McFadden B A, Purohit S (1977) Itaconate, an isocitrate lyase-directed inhibitor in Pseudomonas indigofera. J Bacteriol 131:136-144.
31. Hillier S, Charnetzky W T (1981) Glyoxylate bypass enzymes in Yersinia species and multiple forms of isocitrate lyase in Yersinia pestis. J Bacteriol 145:452-458.
32. Sharma V, et al. (2000) Structure of isocitrate lyase, a persistence factor of Mycobacterium tuberculosis. Nat Struct Biol 7:663-638.
33. Höner Zu Bentrup K, Miczak A, Swenson D L, Russell D G (1999) Characterization of activity and expression of isocitrate lyase in Mycobacterium avium and Mycobacterium tuberculosis. J Bacteriol 181:7161-7167.
34. Hou J Y, Graham J E, Clark-Curtiss J E (2002) Mycobacterium avium genes expressed during growth in human macrophages detected by selective capture of transcribed sequences (SCOTS). Infect Immun 70:3714-3726.
35. McKinney J D, et al. (2000) Persistence of Mycobacterium tuberculosis in macrophages and mice requires the glyoxylate shunt enzyme isocitrate lyase. Nature 406:735-738.
36. Muñoz-Elías E J, McKinney J D (2005) Mycobacterium tuberculosis isocitrate lyases 1 and 2 are jointly required for in vivo growth and virulence. Nat Med 11:638-644.
37. Russell D G (2001) Mycobacterium tuberculosis: here today, and here tomorrow. Nat Rev Mol Cell Biol 2:569-577.
38. Muñoz-Elías E J, Upton A M, Cherian J, McKinney J D (2006) Role of the methylcitrate cycle in Mycobacterium tuberculosis metabolism, intracellular growth, and virulence. Mol Microbiol 60:1109-1122.
39. Savvi S, et al. (2008) Functional characterization of a vitamin B12-dependent methylmalonyl pathway in Mycobacterium tuberculosis: implications for propionate metabolism during growth on fatty acids. J Bacteriol 190:3886-3895.
40. Russell D G, et al. (2010) Mycobacterium tuberculosis wears what it eats. Cell Host Microbe 8:68-76.
41. Knowles R G, Moncada S (1994) Nitric oxide synthases in mammals. Biochem J 298 (Pt 2):249-258.
42. Bocchini V, et al. (1992) An immortalized cell line expresses properties of activated microglial cells. J Neurosci Res 31:616-621.
43. Raschke W C, Baird S, Ralph P, Nakoinz I (1978) Functional macrophage cell lines transformed by Abelson leukemia virus. Cell 15:261-267.
44. Giard D J, et al. (1973) In vitro cultivation of human tumors: establishment of cell lines derived from a series of solid tumors. J Natl Cancer Inst 51:1417-1423.
45. Graham F L, Smiley J, Russell W C, Nairn R (1977) Characteristics of a human cell line transformed by DNA from human adenovirus type 5. J Gen Virol 36:59-74.
46. Smal C, et al. (2006) Identification of in vivo phosphorylation sites on human deoxycytidine kinase. Role of Ser-74 in the control of enzyme activity. J Biol Chem 281:4887-4893.
47. Steinhäuser C, et al. (2013) Lipid-Labeling Facilitates a Novel Magnetic Isolation Procedure to Characterize Pathogen-Containing Phagosomes. Traffic 14:321-336.
48. Song H, Sandie R, Wang Y, Andrade-Navarro M A, Niederweis M (2008) Identification of outer membrane proteins of Mycobacterium tuberculosis. Tuberculosis (Edinb) 88:526-544.
Vilella A J, et al. (2009) EnsemblCompara GeneTrees: Complete, duplication-aware phylogenetic trees in vertebrates. Genome Res 19:327-35.
49. Katoh K, Toh H (2008) Recent developments in the MAFFT multiple sequence alignment program. Brief Bioinformatics 9:286-298.
50. Katoh K, Misawa K, -ichi Kuma K, Miyata T (2002) MAFFT: a novel method for rapid multiple sequence alignment based on fast Fourier transform. Nucleic Acids Res 30:3059-3066.
51. Gouet P, Courcelle E, Stuart D I, Métoz F (1999) ESPript: analysis of multiple sequence alignments in PostScript. Bioinformatics 15:305-308.
53. Steiger M G, Blumhoff M L, Mattanovich D, Sauer M (2013) Biochemistry of microbial itaconic acid production. Frontiers in Microbiology 4:23
54. Willke T, Vorlop K D (2001) Biotechnological production of itaconic acid. Appl Microbiol Biotechnol. 56(3-4):289-95.
55. Kuenz A, Gallenmüller Y, Willke T, Vorlop K D (2012) Microbial production of itaconic acid: developing a stable platform for high product concentrations. Appl Microbiol Biotechnol 96:1209-1216
56. Tate B E (1981) Itaconic acid and derivatives. In: Grayson M Eckroth E (eds) Kirk-Othmer Encycl Chem Technol 3, pp 865-873
57. Voll A, Klement T, Gerhards G, Büchs J, Marquardt W (2012). Metabolic modelling of itaconic acid fermentation with Ustilago maydis. Chem. Eng. Trans. 27, 367-372.
58. Parekh S, Vinci V A, Strobel R J (2000) Improvement of microbial strains and fermentation processes. Appl Microbiol Biotechnol. 54(3):287-301.
59. Adrio J L, Demain A L (2010) Recombinant organisms for production of industrial products. Bioeng Bugs. 1(2):116-31.
60. Yahiro K et al. (1995) Breeding of Aspergillus terreus mutant TN-484 for itaconic acid production with high yield. J Ferm Bioeng 79:506-508.
61. Reddy C S, Singh R P (2002) Enhanced production of itaconic acid from corn starch and market refuse fruits by genetically manipulated Aspergillus terreus SKR10. Bioresour Technol. 85(1):69-71.
62. Dwiarti et al. (2002), Journal of Bioscience and Bioengineering, Vol. 94, No. 1, 29-33

1. A method for the production of itaconic acid, comprising
(i) expressing in a non-human host cell a nucleic acid molecule selected from the group consisting of
(a) a nucleic acid molecule having the nucleotide sequence shown in SEQ ID NO:1 or 3;
(b) a nucleic acid molecule encoding a polypeptide having the amino acid sequence shown in SEQ ID NO:2 or 4;
(c) a nucleic acid molecule encoding a fragment of a polypeptide encoded by a nucleic acid molecule of (a) or (b), wherein said fragment has cis-aconitic acid decarboxylase (CAD) activity;
(d) a nucleic acid molecule which is at least 50% identical to a nucleic acid molecule as defined in any one of (a) to (c) and which encodes a polypeptide having CAD activity; and
(e) a nucleic acid molecule, the complementary strand of which hybridizes under stringent conditions to a nucleic acid molecule as defined in any one of (a) to (d) and which encodes a polypeptide having CAD activity; and
(ii) cultivating said host cell.

2. The method of claim 1, wherein said nucleic acid molecule is heterologous to said host cell.

3. The method of claim 1, wherein said host cell is a prokaryotic cell, a yeast cell or a fungal cell.

4. The method of claim 3, wherein said prokaryotic cell is a gram-negative cell or gram-positive cell.

5. The method of claim 4, wherein said gram-negative cell is E. coli.

6. The method of claim 4, wherein said gram-positive cell is B. subtilis or B. megaterium.

7. The method of claim 3, wherein said fungal cell is Aspergillus sp., Yarrowia lipolytica, Ustilago maydis, Ustilago zeae, Candida sp., Rhodotorula sp. or Pseudozyma antarctica.

8. The method of claim 3, wherein said fungal cell is Aspergillus terreus, Aspergillus niger, Aspergillus itaconicus or Aspergillus flavus.

9. The method of claim 3, wherein said fungal cell is a Aspergillus sp. optimized for the production of itaconic acid.

10. The method of claim 3, wherein said fungal cell is Aspergillus terrus MJL05, Aspergillus terreus TN484, Aspergillus terreus NRRL 1960, Aspergillus terreus IMI 282743 or Aspergillus terreus IFO 6365.

11. The method of claim 3, wherein said yeast cell is Saccharomyces cerevisiae, Schizosaccharomyces pombe, Kluyveromyces lactis, Hansenula polymorpha or Pichia pastoris.

12. The method of claim 3, wherein said host cell is modified for industrial application.

13. The method of claim 3, wherein said host cell is optimized for the production of itaconic acid.

14. The method of claim 1, further comprising isolating itaconic acid from said host cell and/or the extracellular medium.

15. The method of claim 1, wherein itaconic acid is obtained.

16. The method of claim 1, wherein itaconic acid is obtained and further processed.

17. The method of claim 1, wherein the produced itaconic acid is further processed.

18. A composition of matter comprising at least 1 g/l itaconic acid and the nucleic acid molecule of claim 1.

19. A non-human host cell comprising a nucleic acid molecule selected from any of the group comprising:
(a) a nucleic acid molecule having the nucleotide sequence shown in SEQ ID NO:1 or 3;
(b) a nucleic acid molecule encoding a polypeptide having the amino acid sequence shown in SEQ ID NO:2 or 4;
(c) a nucleic acid molecule encoding a fragment of a polypeptide encoded by a nucleic acid molecule of (a) or (b), wherein said fragment has cis-aconitic acid decarboxylase (CAD) activity;
(d) a nucleic acid molecule which is at least 50% identical to a nucleic acid molecule as defined in any one of (a) to (c) and which encodes a polypeptide having CAD activity; and
(e) a nucleic acid molecule, the complementary strand of which hybridizes under stringent conditions to a nucleic acid molecule as defined in any one of (a) to (d) and which encodes a polypeptide having CAD activity.

20. A non-human host cell comprising a polypeptide encoded by a nucleic acid molecule selected from any of the group comprising:
(a) a nucleic acid molecule having the nucleotide sequence shown in SEQ ID NO:1 or 3;
(b) a nucleic acid molecule encoding a polypeptide having the amino acid sequence shown in SEQ ID NO:2 or 4;
(c) a nucleic acid molecule encoding a fragment of a polypeptide encoded by a nucleic acid molecule of (a) or (b), wherein said fragment has cis-aconitic acid decarboxylase (CAD) activity;
(d) a nucleic acid molecule which is at least 50% identical to a nucleic acid molecule as defined in any one of (a) to (c) and which encodes a polypeptide having CAD activity; and
(e) a nucleic acid molecule, the complementary strand of which hybridizes under stringent conditions to a nucleic acid as defined in any one of (a) to (d) and which encodes a polypeptide having CAD activity.

21. A kit for the production of itaconic acid including a nucleic acid molecule as defined in claim 1 or the host cell of claim 19 or 20.

22. Use of a nucleic acid molecule selected from the group consisting of
(a) a nucleic acid molecule having the nucleotide sequence shown in SEQ ID NO:1 or 3;
(b) a nucleic acid molecule encoding a polypeptide having the amino acid sequence shown in SEQ ID NO:2 or 4;
(c) a nucleic acid molecule encoding a fragment of a polypeptide encoded by a nucleic acid molecule of (a) or (b), wherein said fragment has cis-aconitic acid decarboxylase (CAD) activity;
(d) a nucleic acid molecule which is at least 50% identical to a nucleic acid molecule as defined in any one of (a) to (c) and which encodes a polypeptide having CAD activity; and
(e) a nucleic acid molecule, the complementary strand of which hybridizes under stringent conditions to a nucleic acid molecule as defined in any one of (a) to (d) and which encodes a polypeptide having CAD activity; and
for producing itaconic acid.
